(19) United States
(12) Patent Application Publication
(10) Pub. No.: US 2023/0338502 A1
(43) Pub. Date: Oct. 26, 2023

(54) Methods Of Treatment Of High Grade Squamous Intraepithelial Lesion (HSIL)

(71) Applicant: Inovio Pharmaceuticals, Inc., Plymouth Meeting, PA (US)

(72) Inventors: Matthew P. Morrow, Malvern, PA (US); Kimberly A. Kraynyak, Norristown, PA (US); Albert J. Sylvester, Croydon, PA (US); Naseem Prostak, Easton, PA (US); Laurent M. Humeau, San Diego, CA (US)

(21) Appl. No.: 18/300,588

(22) Filed: Apr. 14, 2023

Related U.S. Application Data
(60) Provisional application No. 63/333,331, filed on Apr. 21, 2022.

(51) Int. Cl. A61K 39/12; A61P 15/00; A61P 31/20; C12Q 1/70; C12Q 1/6869

(52) U.S. Cl. CPC A61K 39/12; A61P 15/00; A61P 31/20; C12Q 1/708; C12Q 1/6869; C12Q 2600/178

(57) ABSTRACT

Described are methods of treating human papillomavirus (HPV) type 16- or HPV type 18-related High-grade Squamous Intraepithelial Lesion (HSIL) of the cervix. The described methods comprise evaluating one or more biological samples for the presence of miRNAs and isomiRs thereof.

### CROSS REFERENCE TO RELATED APPLICATIONS

This application claims the benefit of U.S. Provisional Patent Application No. 63/333,331, filed Apr. 21, 2022, the disclosure of which is incorporated herein by reference it its entirety.

### SEQUENCE LISTING

This application contains a Sequence Listing which is submitted electronically as an XML formatted sequence listing with file name “104409_000848_Sequence_Listing.xml” and a creation date of Apr. 12, 2023, and a size of 7000 bytes. The Sequence Listing submitted electronically is part of the specification and is incorporated by reference in its entirety.

### TECHNICAL FIELD

Provided are methods of treating human papillomavirus (HPV) type 16- or HPV type 18-related High-grade Squamous Intraepithelial Lesion (HSIL) of the cervix. The provided methods comprise evaluating one or more biological samples for the presence of miRNAs and isomiRs thereof.

### BACKGROUND

MicroRNAs (miRNAs) are a class of endogenous small noncoding RNA, typically ranging from 19-24 nucleotides. When loaded into the microribonucleoprotein (miRNP) or RNA induced silencing complex (RISC), miRNAs bind to miRNA recognition elements (MREs) on mRNA targets, exerting gene regulation through post-transcriptional and/translational mechanisms.

To date, no miRNA profile yet exists for the purpose of predicting response to HPV type-specific treatment of HPV16- or HPV18-related HSIL.

### SUMMARY

Provided herein are methods of treating human papillomavirus (HPV) type 16- or HPV type 18-related high grade cervical intraepithelial lesion (HSIL), said method comprising: (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive.

In certain embodiments, the presence of the miRNA is determined by RNA sequencing.

In further embodiments, the biological sample is a plasma sample.

In still further embodiments, the plasma sample is isolated from the subject prior to administration of VGX-3100.

In additional embodiments, the HPV type 16- or HPV type 18-related HSIL of the cervix is determined by a biopsy.

In some embodiments, the VGX-3100 is administered to the subject by intramuscular injection followed by electroporation. In certain embodiments, the VGX-3100 is administered to the subject at a dose of 6 mg. In further embodiments, the VGX-3100 is administered to the subject three times over the course of 12 weeks. In still further embodiments, the VGX-3100 is formulated at a concentration of 6 mg/ml in 150 mM sodium chloride and 15 mM sodium citrate.

In certain embodiments, administration of VGX-3100 results in virologic clearance of HPV-16 and/or HPV-18 and histopathologic regression of cervical HSIL. In further embodiments, administration of VGX-3100 results in histopathologic regression of cervical HSIL. In still further embodiments, administration of VGX-3100 results in virologic clearance of HPV-16 and/or HPV-18. In certain embodiments, administration of VGX-3100 results in complete histopathologic regression of cervical HSIL to normal. In further embodiments, administration of VGX-3100 results in complete histopathologic regression of cervical HSIL to normal and virologic clearance of HPV-16 and/or HPV-18. In still further embodiments, administration of VGX-3100 results in histopathologic nonprogression. In certain embodiments, administration of VGX-3100 results in clearance of HPV-16 and/or HPV-18 infection from noncervical anatomic locations. In further embodiments, administration of VGX-3100 results in improved humoral and cellular immune response to VGX-3100 following a third administration of VGX-3100 and at 36 weeks following administration of VGX-3100 as assessed relative to baseline.

In certain embodiments, the result of VGX-3100 administration is evaluated at 36 weeks following administration of VGX-3100.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

When a range of numerical values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods, which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

### Certain Terminology

As used herein, the term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

As used herein, the term “at least one” means “one or more.”

As used herein, the term “subject” as used herein refers to any animal, but in particular humans. Thus, the methods are applicable to human and nonhuman animals, although preferably used most preferably with humans. “Subject” and “patient” are used interchangeably herein.

As used herein, the term “comprising” is intended to include examples encompassed by the terms “consisting essentially of” and “consisting of”; similarly, the term “consisting essentially of” is intended to include examples encompassed by the term “consisting of.”

As used herein, “treating” and like terms refer to reducing the severity and/or frequency of human papillomavirus (HPV) type 16- or HPV type 18-related high grade cervical intraepithelial lesion (HSIL) of the cervix symptoms for example and cervical high grade squamous intraepithelial lesions (HSIL) lesions; eliminating HPV type 16 or HPV type 18 infection symptoms, especially HSIL lesions; and/or clearing HPV type 16 or HPV type 18 virus from the subject; and/or resolution to cervical low grade squamous intraepithelial lesions (LSIL) or normal tissue.

As used herein, the term “coding sequence” or “encoding nucleic acid” may mean refers to the nucleic acid (RNA or DNA molecule) that comprise a nucleotide sequence which encodes a polypeptide. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides, e.g., an IgE leader sequence.

As used herein, the term “nucleic acid” or “oligonucleotide” or “polynucleotide” may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, the term “operably linked” may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function,

As used herein, the term “promoter” may mean a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

As used herein, the term “vector” may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

As used herein, the term “adverse event” (AE) is defined according to the Common Toxicity Criteria for Adverse Events (CTCAE) Version 4.03 grading scale. The investigator graded the laboratory AEs and clinical AEs with respect to the following levels of severity as per CTCAE Version 4.03 for applicable subject populations: grade 1 (mild), grade 2 (moderate), grade 3 (severe), grade 4 (potentially life-threatening), and grade 5 (death).

As used herein, the term “isomiR” refers to isoforms of canonical (mature) miRNAs. IsomiRs may have the following variations from the canonical miRNA: (1) addition and/or deletion of nucleotide(s) at the 5′-end of the canonical miRNA; (2) addition and/or deletion of nucleotide(s) at the 3′-end of canonical miRNA; (3) addition and/or deletion of nucleotide(s) at both 5′- and 3′-ends of the canonical miRNA; and (4) nucleotide substitution within the sequence of the canonical miRNA.

As used herein, the term “responder” refers to study subjects who met the primary endpoint of HSIL lesion regression to LSIL or lower with concomitant elimination of HPV16/HPV18 infection.

As used herein, the term “Biomarker Positive” refers to study subjects that the biomarker signature (e.g. the RF_21_20 model) predicts to be a Responder as defined above prior to VGX-3100 or placebo administration.

As used herein, the term “PPV” refers to the number of biomarker-positive subjects who are responders/the number of biomarker-positive subjects.

As used herein, the term “NPV” refers to the number of biomarker-negative subjects who are responders/the number of biomarker-negative subjects.

As used herein, the term “accuracy” refers to the number biomarker-positive subjects who are responders plus the number biomarker-negative subjects who are non-responders/the number of subjects.

As used herein, the term “Sensitivity” refers to the number of biomarker-positive subjects who are responders/the number of responders.

As used herein, the term “specificity” refers to the number of biomarker-negative subjects who are non-responders/the number non-responders.

As used herein, the term “placebo” means administration of a pharmaceutical composition that does not include VGX-3100.

### Methods of Treatment and Diagnosis

Provided herein are methods of treating human papillomavirus (HPV) type 16- or HPV type 18-related high grade cervical intraepithelial lesion (HSIL), said method comprising, consisting of, or consisting essentially of: (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive.

Also provided herein is VGX-3100 for use in a method of treating human papillomavirus (HPV) type 16- or HPV type 18-related high grade cervical intraepithelial lesion (HSIL), said method comprising, consisting of, or consisting essentially of: (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as a biomarker positive.

Also provided herein are uses of VGX-3100 in the manufacture of a medicament for treating human papillomavirus (HPV) type 16- or HPV type 18-related high grade cervical intraepithelial lesion (HSIL), said method comprising, consisting of, or consisting essentially of: (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive.

Provided herein are methods of treating human papillomavirus (HPV) type 16- or HPV type 18-related high grade cervical intraepithelial lesion (HSIL), said method comprising, consisting of, or consisting essentially of: (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) withholding administration of a therapeutically effective amount of VGX-3100 to said subject if the subject is categorized as biomarker negative. In certain embodiments, the method further comprises treating said subject with loop electrosurgical excision procedure [LEEP]/large loop excision of the transformation zone [LLETZ], laser ablation, or conization.

In certain embodiments, the miRNA signature is determined using a model that possesses the ability to predict regression of cervical HSIL and clearance of HPV16/18 infection following VGX-3100 treatment as demonstrated by the fact that accuracy, PPV, NPV, sensitivity and specificity are all superior when this model is applied to the treated group compared to placebo.

In certain embodiments, the presence of the miRNA is determined by RNA sequencing.

In further embodiments, the biological sample is a plasma sample.

In still further embodiments, the plasma sample is isolated from the subject prior to administration of VGX-3100.

In additional embodiments, the HPV type 16- or HPV type 18-related HSIL of the cervix is determined by a biopsy.

In some aspects of the present disclosure, the methods comprise evaluating one or more biological sample. In certain embodiments, the methods comprising evaluating one biological sample. In certain embodiments, the methods comprising evaluating two biological samples. In certain embodiments, the methods comprising evaluating three biological sample. In certain embodiments, the methods comprising evaluating four biological samples. In certain embodiments, the methods comprising evaluating five biological samples.

Also provided herein are methods of selecting a subject as a candidate for treatment with VGX-3100, said method comprising, consisting of, or consisting essentially of: (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as a candidate for treatment with VGX-3100 based on the miRNA signature. In certain embodiments, the method further comprises administering a therapeutically effective amount of VGX-3100 to said subject is the subject is categorized as a candidate for treatment with VGX-3100.

Also provided herein are methods of eliminating a subject as a candidate for treatment with VGX-3100, said method comprising, consisting of, or consisting essentially of: (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as not being a candidate for treatment with VGX-3100 based on the miRNA signature. In certain embodiments, the method further comprises withholding administration of a therapeutically effective amount of VGX-3100 to said subject is the subject is not categorized as a candidate for treatment with VGX-3100. In certain embodiments, the method further comprises treating said subject with loop electrosurgical excision procedure [LEEP]/large loop excision of the transformation zone [LLETZ], laser ablation, or conization.

In certain embodiments, administration of VGX-3100 results in virologic clearance of HPV-16 and/or HPV-18 and histopathologic regression of cervical HSIL. In further embodiments, administration of VGX-3100 results in histopathologic regression of cervical HSIL. In still further embodiments, administration of VGX-3100 results in virologic clearance of HPV-16 and/or HPV-18. In certain embodiments, administration of VGX-3100 results in complete histopathologic regression of cervical HSIL to normal. In further embodiments, administration of VGX-3100 results in complete histopathologic regression of cervical HSIL to normal and virologic clearance of HPV-16 and/or HPV-18. In still further embodiments, administration of VGX-3100 results in histopathologic nonprogression. In certain embodiments, administration of VGX-3100 results in clearance of HPV-16 and/or HPV-18 infection from noncervical anatomic locations. In further embodiments, administration of VGX-3100 results in improved humoral and cellular immune response to VGX-3100 following a third administration of VGX-3100 and at 36 weeks following administration of VGX-3100 as assessed relative to baseline.

Also provided herein are methods of improving virologic clearance of HPV-16 and/or HPV-18 and histopathologic regression of cervical HSIL in a subject comprising, consisting of, or consisting essentially of (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive. In certain embodiments, the improvement in virologic clearance of HPV-16 and/or HPV-18 and histopathologic regression of cervical HSIL is relative to administration of a placebo to a subject or a population of subjects. In certain embodiments, the improvement in virologic clearance of HPV-16 and/or HPV-18 and histopathologic regression of cervical HSIL is relative to no treatment of a subject or a population of subjects. In certain embodiments, the improvement in virologic clearance of HPV-16 and/or HPV-18 and histopathologic regression of cervical HSIL is relative to treatment of a subject of population of subjects with the standard of care.

Also provided herein are methods of improving histopathologic regression of cervical HSIL in a subject comprising, consisting of, or consisting essentially of (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive. In certain embodiments, the improvement in histopathologic regression of cervical HSIL is relative to administration of a placebo to a subject or a population of subjects. In certain embodiments, the improvement in histopathologic regression of cervical HSIL is relative to no treatment of a subject or a population of subjects. In certain embodiments, the improvement in histopathologic regression of cervical HSIL is relative to treatment of a subject of population of subjects with the standard of care.

Also provided herein are methods of improving virologic clearance of HPV-16 and/or HPV-18 in a subject comprising, consisting of, or consisting essentially of (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive. In certain embodiments, the improvement in virologic clearance of HPV-16 and/or HPV-18 is relative to administration of a placebo to a subject or a population of subjects. In certain embodiments, the improvement in virologic clearance of HPV-16 and/or HPV-18 is relative to no treatment of a subject or a population of subjects. In certain embodiments, the improvement in virologic clearance of HPV-16 and/or HPV-18 is relative to treatment of a subject of population of subjects with the standard of care.

Also provided herein are methods of achieving complete histopathologic regression of cervical HSIL to normal in a subject comprising, consisting of, or consisting essentially of (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive. In certain embodiments, the achievement of complete histopathologic regression of cervical HSIL to normal is relative to administration of a placebo to a subject or a population of subjects. In certain embodiments, the achievement of complete histopathologic regression of cervical HSIL is relative to no treatment of a subject or a population of subjects. In certain embodiments, the achievement of complete histopathologic regression of cervical HSIL is relative to treatment of a subject of population of subjects with the standard of care.

Also provided herein are methods of achieving complete histopathologic regression of cervical HSIL to normal and virologic clearance of HPV-16 and/or HPV-18 in a subject comprising, consisting of, or consisting essentially of (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive. In certain embodiments, the achievement of complete histopathologic regression of cervical HSIL to normal and virologic clearance of HPV-16 and/or HPV-18 is relative to administration of a placebo to a subject or a population of subjects. In certain embodiments, the achievement of complete histopathologic regression of cervical HSIL to normal and virologic clearance of HPV-16 and/or HPV-18 is relative to no treatment of a subject or a population of subjects. In certain embodiments, the achievement of complete histopathologic regression of cervical HSIL to normal and virologic clearance of HPV-16 and/or HPV-18 is relative to treatment of a subject of population of subjects with the standard of care.

Also provided herein are methods of improving histopathologic nonprogression in a subject comprising, consisting of, or consisting essentially of (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive. In certain embodiments, the improvement in histopathologic nonprogression is relative to administration of a placebo to a subject or a population of subjects. In certain embodiments, the improvement in histopathologic nonprogression is relative to no treatment of a subject or a population of subjects. In certain embodiments, the improvement in histopathologic nonprogression is relative to treatment of a subject of population of subjects with the standard of care.

Also provided herein are methods of improving clearance of HPV-16 and/or HPV-18 infection from noncervical anatomic locations in a subject comprising, consisting of, or consisting essentially of (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive. In certain embodiments, the improvement in clearance of HPV-16 and/or HPV-18 infection from noncervical anatomic locations is relative to administration of a placebo to a subject or a population of subjects. In certain embodiments, the improvement in clearance of HPV-16 and/or HPV-18 infection from noncervical anatomic locations is relative to no treatment of a subject or a population of subjects. In certain embodiments, the improvement in clearance of HPV-16 and/or HPV-18 infection from noncervical anatomic locations is relative to treatment of a subject of population of subjects with the standard of care.

Also provided herein are methods of improving humoral and cellular immune response to VGX-3100 following a third administration of VGX-3100 and at 36 weeks following administration of VGX-3100 as assessed relative to baseline in a subject comprising, consisting of, or consisting essentially of (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, and hsa.miR.744.5p; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive. In certain embodiments, the improvement in clearance of HPV-16 and/or HPV-18 infection from noncervical anatomic locations is relative to administration of a placebo to a subject or a population of subjects. In certain embodiments, the improvement in humoral and cellular immune response to VGX-3100 following a third administration of VGX-3100 and at 36 weeks following administration of VGX-3100 as assessed relative to baseline is relative to no treatment of a subject or a population of subjects. In certain embodiments, the improvement in humoral and cellular immune response to VGX-3100 following a third administration of VGX-3100 and at 36 weeks following administration of VGX-3100 as assessed relative to baseline is relative to treatment of a subject of population of subjects with the standard of care.

In certain embodiments, the result of VGX-3100 administration or the improvement achieved by VGX-3100 administration is evaluated at 36 weeks following administration of VGX-3100.

### Routes of Administration and Pharmaceutical Compositions

VGX-3100 may be delivered using any of several well-known technologies including DNA injection (also referred to as DNA vaccination), recombinant vectors such as recombinant adenovirus, recombinant adenovirus related virus and recombinant vaccinia.

Routes of administration include, but are not limited to, intramuscular, intranasally, intraperitoneal, intradermal, subcutaneous, intravenous, intraarterially, intraocularly and oral as well as topically, transdermally, by inhalation or suppository or to mucosal tissue such as by lavage to vaginal, rectal, urethral, buccal and sublingual tissue. Preferred routes of administration include intramuscular, intraperitoneal, intradermal and subcutaneous injection. Genetic constructs may be administered by means including, but not limited to, electroporation methods and devices, traditional syringes, needleless injection devices, or “microprojectile bombardment gone guns”. In some embodiments, the VGX-3100 is administered to the subject by intramuscular injection. In certain embodiments, the VGX-3100 is administered to the subject by intramuscular injection followed by electroporation.

Examples of electroporation devices and electroporation methods preferred for facilitating delivery of the DNA vaccines, include those described in U.S. Pat. No. 7,245,963 by Draghia-Akli, et al, U.S. Patent Pub. 2005/0052630 submitted by Smith, et al., the contents of which are hereby incorporated by reference in their entirety. Also preferred, are electroporation devices and electroporation methods for facilitating delivery of the DNA vaccines provided in co-pending and co-owned U.S. patent application Ser. No. 11/874072, filed Oct. 17, 2007, which claims the benefit under 35 USC 1 19(e) to U.S. Provisional Applications Ser. Nos. 60/852,149, filed Oct. 17, 2006, and 60/978,982, filed Oct. 10, 2007, all of which are hereby incorporated in their entirety. In certain embodiments, the electroporation device is a CELLECTRA®-5P device.

The following is an example of an embodiment using electroporation technology and is discussed in more detail in the patent references discussed above: electroporation devices can be configured to deliver to a desired tissue of a mammal a pulse of energy producing a constant current similar to a preset current input by a user. The electroporation device comprises an electroporation component and an electrode assembly or handle assembly. The electroporation component can include and incorporate one or more of the various elements of the electroporation devices, including: controller, current waveform generator, impedance tester, waveform logger, input element, status reporting element, communication port, memory component, power source, and power switch. The electroporation component can function as one element of the electroporation devices, and the other elements are separate elements (or components) in communication with the electroporation component. In some embodiments, the electroporation component can function as more than one element of the electroporation devices, which can be in communication with still other elements of the electroporation devices separate from the electroporation component. The use of electroporation technology to deliver the improved HPV vaccine is not limited by the elements of the electroporation devices existing as parts of one electromechanical or mechanical device, as the elements can function as one device or as separate elements in communication with one another. The electroporation component is capable of delivering the pulse of energy that produces the constant current in the desired tissue and includes a feedback mechanism. The electrode assembly includes an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism can receive the measured impedance and can adjust the pulse of energy delivered by the electroporation component to maintain the constant current.

In some embodiments, the plurality of electrodes can deliver the pulse of energy in a decentralized pattern. In some embodiments, the plurality of electrodes can deliver the pulse of energy in the decentralized pattern through the control of the electrodes under a programmed sequence, and the programmed sequence is input by a user to the electroporation component. In some embodiments, the programmed sequence comprises a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

In some embodiments, the feedback mechanism is performed by either hardware or software. Preferably, the feedback mechanism is performed by an analog closed-loop circuit. Preferably, this feedback occurs every 50 μ8, 20 μ8, 10 μ$ or 1 μ8, but is preferably a real-time feedback or instantaneous (i.e., substantially instantaneous as determined by available techniques for determining response time). In some embodiments, the neutral electrode measures the impedance in the desired tissue and communicates the impedance to the feedback mechanism, and the feedback mechanism responds to the impedance and adjusts the pulse of energy to maintain the constant current at a value similar to the preset current. In some embodiments, the feedback mechanism maintains the constant current continuously and instantaneously during the delivery of the pulse of energy.

In some embodiments, the nucleic acid molecule is delivered to the cells in conjunction with administration of a polynucleotide function enhancer or a genetic vaccine facilitator agent. Polynucleotide function enhancers are described in U.S. Pat. Nos. 5,593,972, 5,962,428 and International Application Serial Number PCT/US94/00899 filed Jan. 26, 1994, which are each incorporated herein by reference. The co-agents that are administered in conjunction with nucleic acid molecules may be administered as a mixture with the nucleic acid molecule or administered separately simultaneously, before or after administration of nucleic acid molecules. In addition, other agents which may function transfecting agents and/or replicating agents and/or inflammatory agents and which may be co-administered with a GVF include growth factors, cytokines and lymphokines such as a-interferon, gamma-interferon, GM-CSF, platelet derived growth factor (PDGF), TNF, epidermal growth factor (EGF), IL-1, IL-2, IL-4, IL-6, IL-10, IL-12 and IL-15 as well as fibroblast growth factor, surface active agents such as immune-stimulating complexes (ISCOMS), Freunds incomplete adjuvant, LPS analog including monophosphoryl Lipid A (WL), muramyl peptides, quinone analogs and vesicles such as squalene and squalene, and hyaluronic acid may also be used administered in conjunction with the genetic construct In some embodiments, an immunomodulating protein may be used as a GVF. In some embodiments, the nucleic acid molecule is provided in association with PLG to enhance delivery/uptake.

The pharmaceutical compositions according to the present invention comprise about 1 nanogram to about 2000 micrograms of DNA. In some preferred embodiments, pharmaceutical compositions according to the present invention comprise about 5 nanogram to about 1000 micrograms of DNA. In some preferred embodiments, the pharmaceutical compositions contain about 10 nanograms to about 800 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms of DNA. In some preferred embodiments, the pharmaceutical compositions contain about 100 to about 200 microgram DNA. In certain embodiments, the VGX-3100 is formulated at a concentration of 6 mg/ml.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. In some preferred embodiments, the pharmaceutical compositions contain about 100 to about 200 microgram DNA. In certain embodiments, the VGX-3100 is formulated at a concentration of 6 mg/ml in 150 mM sodium chloride and 15 mM sodium citrate.

In certain embodiments, the VGX-3100 is administered to the subject at a dose of 6 mg. In further embodiments, the VGX-3100 is administered to the subject three times over the course of 12 weeks. In still further embodiments, a first dose of VGX-3100 is administered on Day 0, the second dose of VGX-3100 is administered at Week 4, and the third dose of VGX-3100 is administered at Week 12.

### EXAMPLES

The examples and embodiments described herein are for illustrative purposes only and various modifications or changes suggested to persons skilled in the art are to be included within the spirit and purview of this application and scope of the appended claims.

### Example 1

A Prospective, Randomized, Double-Blind, Placebo-Controlled Phase 3 Study of VGX-3100 Delivered Intramuscularly Followed by Electroporation (EP) With CELLECTRA™ 5PSP for the Treatment of HPV-16 and/or HPV-18 Related High-Grade Squamous Intraepithelial Lesion (HSIL) of the Cervix (REVEAL 1 Trial; Randomized Evaluation of VGX-3100 and Electroporation for the Treatment of Cervical HSIL) (Study HPV-301)

A non-limiting example of a prospective, randomized, double-blind, placebo-controlled study to demonstrate the safety and efficacy of VGX-3100 followed by EP in women with cervical HSIL associated with HPV-16 and/or HPV-18 is provided herein. The primary endpoint was histopathological regression of cervical HSIL and virologic clearance of HPV-16 and/or HPV-18 at Week 36. A placebo-controlled design was selected for this clinical trial because it provided scientific rigor to distinguish a treatment effect, particularly in cervical HSIL for which spontaneous regression could occur.

### Clinical Trial Objectives and Endpoints:

Primary Objective and Endpoint. The primary objective of the study was to determine the efficacy of VGX-3100 compared with placebo with respect to combined histopathologic regression of cervical HSIL and virologic clearance of HPV-16 and/or HPV-18. The primary objective endpoint was evaluated by the proportion of subjects with no evidence of cervical HSIL on histology (i.e., biopsy or excisional treatment) and no evidence of HPV-16 and/or HPV-18 in cervical samples by type-specific HPV testing at Week 36 visit.

Secondary Objectives and Endpoints. The first secondary objective of the study was to evaluate the safety and tolerability of VGX-3100 delivered IM followed by EP with CELLECTRA™ 5PSP. The endpoints of the first secondary objective were evaluated by (a) incidence and severity of local and systemic events for 7 and 28 days following each investigational treatment and for the duration of the clinical trial (through Week 88 visit) and (b) incidence and severity of all AEs including SAEs (e.g., Suspected Unexpected Serious Adverse Reaction [SUSAR], Unanticipated Adverse Device Effect [UADE], and other unexpected AEs) for the duration of the clinical trial (through Week 88 visit).

The second secondary objective of the study was to determine VGX-3100 efficacy compared with placebo as measured by histopathologic regression of cervical HSIL. The endpoint of the second secondary objective was evaluated by the proportion of subjects with no evidence of cervical HSIL on histology (i.e., biopsies or excisional treatment) at Week 36 visit

The third secondary objective of the study was to determine VGX-3100 efficacy compared with placebo as measured by virologic clearance of HPV-16 and/or HPV-18. The endpoint of the third secondary objective was evaluated by the proportion of subjects with no evidence of HPV-16 and/or HPV-18 in cervical samples by type-specific HPV testing at Week 36 visit.

The fourth secondary objective of the study was to determine VGX-3100 efficacy compared with placebo as measured by complete histopathologic regression of cervical HSIL to normal. The endpoint of the fourth secondary objective was evaluated by the proportion of subjects with no evidence of Low-Grade Squamous Intraepithelial Lesion (LSIL) or High-Grade Squamous Intraepithelial Lesion (HSIL) (i.e., no evidence of Cervical Intraepithelial Neoplasia [CIN] 1, CIN2, or CIN3) on histology (i.e., biopsies or excisional treatment) at Week 36 visit.

The fifth secondary objective of the study was to determine VGX-3100 efficacy compared with placebo as measured by both complete histopathologic regression of cervical HSIL to normal and virologic clearance of HPV-16 and/or HPV-18. The endpoint of the fifth secondary objective was evaluated by the proportion of subjects with no evidence of LSIL or HSIL (i.e., no evidence of CIN1, CIN2, or CIN3) on histology (i.e., biopsies or excisional treatment) and no evidence of HPV-16 and/or HPV-18 by type-specific HPV testing at Week 36 visit.

The sixth secondary objective of the study was to determine the efficacy of VGX-3100 compared with placebo as measured by histopathologic nonprogression. The endpoint of the sixth secondary objective was evaluated by the proportion of subjects with no progression of cervical HSIL to cervical carcinoma from baseline on histology (i.e., biopsies or excisional treatment) at Week 36 visit.

The seventh secondary objective of the study was to describe the clearance of HPV-16 and/or HPV-18 infection from noncervical anatomic locations. The endpoint of the seventh secondary objective was evaluated by the proportion of subjects who have cleared HPV-16 and/or HPV-18 on specimens from noncervical anatomic locations (oropharynx, vagina, and intra-anal) at Week 36 visit.

The eighth secondary objective of the study was to determine the humoral and cellular immune response to VGX-3100 compared with placebo at post dose three (3) and Week 36 visit as assessed relative to baseline. The endpoints of the eighth secondary objective were evaluated by: a) the levels of serum anti-HPV-16 and anti HPV-18 antibody concentrations at Weeks 15 and 36 visits; b) IFN-γ ELISpot response magnitudes at baseline and Weeks 15 and 36 visits; and c) flow cytometry response magnitudes at baseline and Week 15 visits.

Exploratory Objectives and Endpoints. The first exploratory objective of the study was to evaluate tissue immune responses to VGX-3100 in cervical samples. The endpoint of the first exploratory objective was assessment of markers including but not limited to CD8+ and FoxP3+ infiltrating cells. Additional assessments could include visualization of granulysin, perforin, CD137, CD103, and PD-L1 in cervical tissue as sample allowed.

The second exploratory objective of the study was to describe association of miRNA profiles, DNA methylation profile, previous colposcopy, cytology, and HPV testing results with Week 36 histologic regression. The endpoint of the second exploratory objective was evaluated by colposcopy, cytology, HPV test results (Weeks 8, 15, and 28 visits), miRNA profile (baseline and Week 8), and DNA methylation profile (baseline and Week 15) in conjunction with histologic regression of cervical HSIL at Week 36 visit.

The third exploratory objective of the study was to describe the durability of virologic clearance of HPV-16 and/or HPV-18 for subjects treated with VGX-3100 compared with those treated with placebo. The endpoint of the third exploratory objective was evaluated by the proportion of subjects with no evidence of HPV-16 and/or HPV-18 by type specific HPV testing at Weeks 62 and 88 visits.

The fourth exploratory objective of the study was to describe the patient-reported outcomes for subjects treated with VGX-3100. Patient-reported outcome questionnaires were self-administered at baseline, Weeks 4 and 12, 8-14 days following each dose, and at Weeks 28, 36, 40, and 88 by enrolled subjects.

The fifth exploratory objective of the study was to Determine whether a tissue-based score derived using immunologic markers (immunoscore) at baseline was predictive for histological and virological response to VGX-3100 at Week 36. The endpoint of the fifth exploratory objective was evaluated by immunoscore results for VGX-3100 treated subjects in conjunction with histological and virological outcomes at Week 36.

### Methodology

This was a prospective, randomized, double-blind, placebo-controlled clinical trial to determine the efficacy, safety, and tolerability of intramuscular (IM) VGX-3100 injection followed by EP delivered with the CELLECTRA™ 5PSP device in adult women with histologically confirmed cervical HSIL (CIN2 and CIN3) associated with HPV-16 and/or HPV-18.

The clinical trial consisted of a screening period (up to 10 weeks), treatment and follow-up period (36 weeks), and long-term follow-up period (52 weeks). The total duration of participation in the clinical trial for each subject was up to 98 weeks.

Approximately 198 eligible subjects were to be randomly assigned to receive either 6 mg (in 1 mL) VGX-3100 or placebo (ratio 2:1), IM followed by EP. Subjects were to be randomly assigned in a stratified manner according to: 1) CIN severity observed in the biopsy specimens at screening (CIN2 vs. CIN3), 2) body mass index (BMI) category (≤25 kg/m² versus >25 kg/m2) on Day 0, and 3) age category (<25 years versus ≥25 years) on Day 0. To ensure CIN2 disease was not overrepresented in the clinical trial, the percentage of subjects enrolled with CIN2 were to not exceed 50% of the total enrolled.

### Screening Period

All screening evaluations were to be completed within 10 weeks of first dose of clinical trial treatment (Day 0), except for the safety laboratory assessments, which were to be performed within 45 days prior to Day 0.

### Diagnosis and Main Criteria for Inclusion and Exclusion

To be eligible for the clinical trial, subjects were to be at least 18 years of age, provide consent to participate, and have cervical biopsy/biopsies of the cervical lesion(s) at the time of screening. Slides of the biopsy were to be sent to a central pathology laboratory for review by the PAC in a blinded manner to establish the presence of cervical HSIL within screening. In order to be eligible for randomization, the PAC was to assign the histologic diagnosis of cervical HSIL. Subjects were to also have a cervical ThinPrep™ specimen test positive for HPV-16 and/or HPV-18 by coba™ HPV test. Subjects were required to provide informed consent for use of any information collected prior to consenting and before any additional clinical trial-specific procedures could be performed.

### Inclusion Criteria

Subjects were eligible for inclusion in the clinical trial if they met all of the following criteria: women aged 18 years and above and met the minimum age of consent per local regulations; subject had confirmed cervical infection with HPV types 16 and/or 18 at screening by cobas™ HPV test; subject had cervical tissue specimen/slides provided to clinical trial PAC for diagnosis collected within 10 weeks prior to anticipated date of first dose of clinical trial drug; subject had histologic evidence of cervical HSIL as confirmed by PAC at screening; subject understood, agreed, and was able to comply with the requirements of the protocol and was willing and able to provide voluntary consent to participate and sign a consent form prior to clinical trial-related activities; subject was an appropriate candidate for the protocol-specified procedure (i.e., excision, 4-quadrant biopsy with ECC, or 4-quadrant biopsy) required at Week 36, as judged by the investigator; subject had a satisfactory colposcopy at screening, defined as full visualization of the squamocolumnar junction (type I or II transformation zone) and complete visualization of the upper limit of acetowhite epithelium or suspected CIN disease; subject had a cervical lesion that was accessible for sampling by biopsy instrument (e.g., Mini-Tischler device); subject had a cervical lesion of adequate size to ensure that a visible lesion remained after screening biopsy; subject had to meet one of the following criteria with respect to their reproductive capacity: a) postmenopausal as defined by spontaneous amenorrhea for more than 12 months, b) surgically sterile due to absence of ovaries or due to a bilateral tubal ligation/occlusion that was performed more than 12 months prior to screening, or c) woman of childbearing potential (WOCBP) willing to use a contraceptive method, with failure rate of less than 1% per year when used consistently and correctly, from screening until Week 36; and subject had normal screening ECG or screening ECG with no clinically significant findings, as judged by the investigator.

### Exclusion Criteria

Subjects were to be excluded from participation in this clinical trial if they met any of the following criteria: subject had microscopic or gross evidence of adenocarcinoma in situ (AIS), high grade vulvar, vaginal (inclusive of cervical HPV-related lesions that extended into the vaginal vault), or anal intraepithelial neoplasia or invasive cancer in any histopathologic specimen at screening; subject had cervical lesion(s) that could not be fully visualized on colposcopy due to extension high into cervical canal at screening; subject had history of ECC which showed a potentially untreated carcinoma, untreated HSIL, indeterminate or insufficient for diagnosis (ECC was not required to be performed as part of clinical trial screening); subject had treatment for cervical HSIL within 4 weeks prior to screening; subject was pregnant or breastfeeding or was considering becoming pregnant through Week 36 visit; subject had history of previous therapeutic HPV vaccination (licensed prophylactic HPV vaccines were allowed, e.g., Gardasil™, Cervarix™); subject had any unresolved abnormal clinical screening laboratory values of grade 1 or greater per CTCAE Version 4.03 and deemed clinically significant by the investigator within 45 days prior to Day 0; subject had immunosuppression as a result of underlying illness or treatment including: a) history of or positive serologic test for human immunodeficiency virus (HIV) at screening (performed within 45 days prior to Day 0), b) primary immunodeficiencies, c) long-term use (≥7 days) of oral or parenteral glucocorticoids at a dose of ≥20 mg/day of prednisone equivalent (use of inhaled, otic, and ophthalmic corticosteroids was allowed), d) current or anticipated use of disease modifying doses of antirheumatic drugs (e.g., azathioprine, cyclophosphamide, cyclosporine, methotrexate), and biologic disease modifying drugs such as tumor necrosis factor (TNF)-α inhibitors (e.g., infliximab, adalimumab, or etanercept), e) history of solid organ or bone marrow transplantation, f) any prior history of other clinically significant immunosuppressive or clinically diagnosed autoimmune disease that could have jeopardized the safety of the subject or required therapy that could have interfered with clinical trial assessments or endpoint evaluation, or otherwise impacted the validity of the clinical trial results, g) malnourished (e.g., medically significant unintentional weight loss, kwashiorkor, or marasmus) based on screening laboratory tests, medical history, and physical examination, per investigator's clinical judgment; subject had received any nonclinical trial-related, non-live vaccine within 2 weeks of dosing; subject had received any nonclinical trial, live vaccine (e.g., measles vaccine) within 4 weeks of dosing; subject currently had or had history of clinically significant, medically unstable disease which, in the judgment of the investigator, could have jeopardized the safety of the subject, interfered with clinical trial assessments or endpoint evaluation, or otherwise impacted the validity of the clinical trial results (e.g., chronic renal failure; angina, myocardial ischemia, or infarction; class 3 or higher congestive heart failure, cardiomyopathy, or clinically significant arrhythmias); subject had malignancy or systemic treatment for malignancy within 2 years of screening (with the exception of curatively treated, localized anogenital cancers and superficial skin cancers which were allowed); subject had acute or chronic bleeding or clotting disorder that would contraindicate IM injections, or use of blood thinners (e.g., anticoagulants or antiplatelet drugs) within 2 weeks of Day 0; subject had history of seizures unless subject was seizure-free for 5 years with the use of one (1) or fewer antiepileptic agents; subject had sustained, manually confirmed, sitting systolic blood pressure >150 mmHg or <90 mmHg or a diastolic blood pressure >95 mmHg at screening or Day 0; subject had resting heart rate <50 beats per minute (unless attributable to athletic conditioning) or >100 beats per minute at screening or Day 0; subject had prior major surgery within 4 weeks of Day 0; subject had participated in an interventional clinical trial with an investigational compound or device within 30 days of signing informed consent; participation in an observational study was permitted; subject had less than two (2) acceptable sites available for IM injection considering the deltoid and anterolateral quadriceps muscles; subject had tattoos, keloids, or hypertrophic scars located within 2 cm of intended treatment site; subject had cardioverter-defibrillator or pacemaker (to prevent a life-threatening arrhythmia) that was located in ipsilateral deltoid injection-site (unless deemed acceptable by a cardiologist); subject had metal implants or implantable medical device within the EP area; subject had active drug or alcohol use or dependence that, in the opinion of the investigator, would interfere with adherence to clinical trial requirements; subject was a prisoner or was compulsorily detained (involuntarily incarcerated) for treatment of either a psychiatric or physical (i.e., infectious disease) illness; subject was an active military service personnel; subject was a clinical trial-related staff or family member of clinical trial-related staff; or subject had any illness or condition that, in the opinion of the investigator, could affect the safety of the subject or the evaluation of any clinical trial endpoint.

### Treatment and Long-Term Follow-Up Periods

Eligible subjects received three (3) 6-mg doses of VGX-3100 refrigerated formulation or placebo, IM (deltoid [preferred site] or anterolateral quadriceps [alternate site]), followed immediately by EP with the CELLECTRA™ 5PSP device. The first clinical trial treatment was administered on Day 0, the second at Week 4, and the third (final) at Week 12. The first clinical trial treatment was given as soon as possible following confirmation of the cervical HSIL diagnosis and HPV-16 and/or HPV-18 status but no more than 10 weeks following collection of the subject's biopsy specimen used for diagnosis by the Pathology Adjudication Committee (PAC) during screening, contemporaneous with the positive testing for HPV-16 and/or HPV 18.

The injection site was assessed by clinical trial personnel prior to and at least 30 minutes after each clinical trial treatment and at 2 to 4 weeks after clinical trial treatment. Participant Diary Cards (PDCs) were distributed to subjects on the day of clinical trial treatment. Subjects were advised to record local and systemic adverse events (AEs) for 7 days in the PDC after each clinical trial treatment. Subjects were followed up by a phone call at 8 to 14 days after each clinical trial treatment for PDC review of AEs and injection-site reactions.

Efficacy assessments included histology (i.e., biopsy or excisional treatment), colposcopy, cytology, and HPV testing at screening and at specified visits on and after Day 0. Digital photographs of the cervix following application of acetic acid were used to document colposcopic exam findings. Tissue to be analyzed for evidence of histopathologic regression was obtained at Week 36 either by excision (e.g., Loop Electrosurgical Excision Procedure [LEEP], Large Loop Excision of Transformation Zone [LLETZ], cold knife conization [CKC]) or by biopsy (4-quadrant biopsy or 4-quadrant biopsy with endocervical curettage [ECC]), based upon the assessment at Week 28 of cytology, high-risk HPV status, and colposcopic findings.

All subjects were to undergo a long-term follow-up planned for safety, cytology, and HPV 16 and/or HPV-18 testing at 6 months and 1 year following the Week 36 histopathologic assessment.

Safety was assessed throughout the clinical trial and included monitoring of local and systemic AEs for 7 days following each clinical trial treatment as noted on the PDC and all adverse events (AEs) including serious AEs (SAEs), unanticipated adverse device effects (UADEs), and other unexpected AEs throughout the clinical trial.

An independent Data and Safety Monitoring Board (DSMB) provided safety oversight. The DSMB was to be scheduled to meet quarterly. The DSMB was responsible for advising the sponsor if there appeared to be a safety issue and if it appeared that the proportion of the subjects with regression in the VGX-3100 group was unacceptably low compared with the placebo group; no formal interim analysis was to be performed for this purpose.

Immunogenicity assessments included humoral and cell-mediated immune responses in response to VGX-3100 treatment in blood samples and evidence of elevated immune responses in the cervical tissue samples.

### Number of Subjects (Planned and Analyzed)

A total of 198 subjects were planned to be randomized to receive either 6 mg VGX-3100 or placebo IM followed by EP in a 2:1 ratio.

A total of 201 subjects were randomly assigned to receive either VGX-3100+EP (138 subjects) or placebo+EP (63 subjects).

The percentage of subjects in each analysis set are summarized in Table 1.

TABLE 1
Analysis Sets

| | VGX-3100 + EP | Placebo + EP | Total |
|---|---|---|---|
| Analysis Sets | (N = 138) | (N = 63) | (N = 201) |
| Intent-to-Treat (ITT) | 138 | 63 | 201 |
| Modified ITT (mITT) | 134 (97.1) | 63 (100) | 197 (98.0) |
| Per Protocol | 124 (89.9) | 60 (95.2) | 184 (91.5) |
| Safety | 136 (98.6) | 63 (100) | 199 (99.0) |

Note:
There were 2 subjects who were randomized to VGX-3100 + EP group but not treated.
Note:
Denominator used in the percentage calculations was randomized subjects in each treatment.
Abbreviations: EP: Electroporation; ITT: Intent-to-Treat; N: Sample Size for the Group; n: Number of Subjects.

### Test Product, Dose and Mode of Administration, Batch Number(s)

### Investigational Product

Common name: VGX-3100

Chemical name: Circular, double stranded, deoxyribonucleic acid consisting of 3782 base pairs for the pGX3001 plasmid and 3824 base pairs for the pGX3002 plasmid.

Distinguishing name: Eukaryotic expression plasmids containing HPV 16 and 18- E6 & E7-encoding transcription unit controlled by a synthetic, CMV promoter, and elements required for replication and selection in E. coli, namely a pUC origin of replication (pUC Ori) and a kanamycin resistance gene (Kan R).

Detailed description: VGX-3100, HPV therapeutic vaccine is a combination of two plasmids in equal quantities (i.e. the 6 mg dose will deliver 3 mg of each pGX3001 and pGX3002 plasmids): a) pGX3001: p16ConE6E7, a plasmid encoding for a synthetic HPV16 consensus E6 and E7 fusion gene (“consensus HPV 16-6&7”) into a pVAX1 backbone (Invitrogen, Carlsbad, CA) under the control of the cytomegalovirus immediate-early (CMV) promoter, and b) pGX3002: p18ConE6E7, a plasmid encoding for a synthetic HPV18 consensus E6 and E7 fusion gene (“consensus HPV 18-6&7”) into a pVAX1 backbone (Invitrogen, Carlsbad, CA) under the control of the cytomegalovirus immediate-early (CMV) promoter. VGX-3100 is described in WO2014/165291, which is incorporated herein by reference in its entirety. The nucleic acid and amino acid consensus sequences are provided in Table 2 and Table 3, respectively.

TABLE 2
VGX-3100 consensus sequences (nucleic acid)

| Consensus | | |
|---|---|---|
| Gene | | SEQ ID |
| Fusion | Nucleic Acid Sequence | NO |
| Consensus | gaattcgccaccatggactggacctggatcctgttcctggtggccgccgccacacgggtgcacagcttcc | 1 |
| HPV 16- | aggacccccaggagageggcagaaagctgcctcagctgtgtaccgagctgcagaccaccatccacgac | |
| 6&7 | atcatcctggagtgtgtgtactgtaagcagcagctgctgaggagagaggtgtacgaccgggacctgtgtat | |
| | cgtgtacagggacggcaatccctacgccgtgtgtgacaagtgcctgaagttctacagcaagatcagcgag | |
| | taccggcactactgctacagectgtacggcaccaccctggagcagcagtacaacaagcccctgtgtgac | |
| | ctgctgatccggtgtatcaactgccagaagcccctgcagagacacctggacaagaagcagcggttccac | |
| | aacatcaggggcagatggaccggcagatgtatgagctgctgccggagcagcagaaccagaagggaga | |
| | cccagctgagaggccggaagagaagaagccacggcgatacccccaccctgcacgagtacatgctgga | |
| | cctgcagcctgagaccaccgatctgtacggctacggccagctgaatgacagcagcgaggaggaggatg | |
| | agatcgacggccctgccggccaggccgagcccgacagagcccactacaacatcgtgaccttttgctgta | |
| | agtgtgacagcaccctgagactgtgcgtgcagagcacccacgtggacatcagaaccctggaggatctgc | |
| | tgatgggcaccctgggcatcgtgtgtcccatctgctcccagaaaccctgatgagcggccgc | |
| Consensus | atggactggacctggatcctgttcctggtggccgctgccacacgggtgcacagcgccagattcg | 2 |
| HPV 18- | aggaccccaccaggagcggetacaagctgcccgatctgtgtaccgagctgaacaccagcctgc | |
| 6&7 | aggacatcgagatcacctgtgtgtactgtaagaccgtgctggagctgaccgaggtgttcgagaa | |
| | ggacctgttcgtggtgtacagggacagcatcccccacgccgcctgccacaagtgtatcgacttct | |
| | acagccggatccgggagctgagacactacagcgacagcgtgtacggcgataccctggagaag | |
| | ctgaccaacaccggcctgtacaacctgctgatccggtgcctgagatgccagaagcccctgctga | |
| | gacacctgaacgagaagcggeggttccacaacatcgccggccactacagaggccagtgccac | |
| | agctgctgtaacagggccaggcaggagagactgcagcggagaagagagacccaggtgagg | |
| | ggcaggaagagaagaagccacggccccaaggccaccctgcaggatatcgtgctgcacctgg | |
| | agccccagaatgagatccccgtggatctgctgggccacggccagctgtccgacagcgaggag | |
| | gagaacgacgagatcgacggcgtgaatcaccagcacctgcctgccagaagagccgagcctca | |
| | gaggcacaccatgctgtgtatgtgctgtaagtgtgaggcccggatcgaactggtggtggagagc | |
| | agcgccgacgacctgagagccttccagcagctgttcctgaacaccctgagcttcgtgtgtccttg | |
| | gtgtgccagccagcagtga | |

TABLE 3
VGX-3100 consensus sequences (amino acid)

| Consensus | | |
|---|---|---|
| Fusion | | SEQ ID |
| Gene | Amino Acid Sequence | NO |
| Consensus | MDWTWILFLVAAATRVHSFQDPQESGRKLPQLCTELQTTIHDIILE | 3 |
| HPV 16- | CVYCKQQLLRREVYDRDLCIVYRDGNPYAVCDKCLKFYSKISEYR | |
| 6&7 | HYCYSLYGTTLEQQYNKPLCDLLIRCINCQKPLQRHLDKKQRFHNI | |
| | RGRWTGRCMSCCRSSRTRRETQLRGRKRRSHGDTPTLHEYMLDL | |
| | QPETTDLYGYGQLNDSSEEEDEIDGPAGQAEPDRAHYNIVTFCCKC | |
| | DSTLRLCVQSTHVDIRTLEDLLMGTLGIVCPICSQKP | |
| Consensus | MDWTWILFLVAAATRVHSARFEDPTRSGYKLPDLCTELNTSLQDI | 4 |
| HPV 18- | EITCVYCKTVLELTEVFEKDLFVVYRDSIPHAACHKCIDFYSRIREL | |
| 6&7 | RHYSDSVYGDTLEKLTNTGLYNLLIRCLRCQKPLLRHLNEKRRFH | |
| | NIAGHYRGQCHSCCNRARQERLQRRRETQVRGRKRRSHGPKATL | |
| | QDIVLHLEPQNEIPVDLLGHGQLSDSEEENDEIDGVNHQHLPARRA | |
| | EPQRHTMLCMCCKCEARIELVVESSADDLRAFQQLFLNTLSFVCP | |
| | WCASQQ | |

The test product, VGX-3100, was provided as a solution containing 6 mg (1:1 mix of SynCon™ HPV-16 E6/E7 and HPV-18 E6/E7 plasmids) in 150 mM sodium chloride and 15 mM sodium citrate. VGX-3100 drug product was presented in clear glass cartridges and was injected IM (deltoid [preferred site] or anterolateral quadriceps [alternate site]), followed immediately by EP with the CELLECTRA™ 5PSP device.

The lot numbers of VGX-3100 used in the clinical trial were 1622-035 and 1816-045.

### Control Product, Dose and Mode of Administration, Batch Number(s):

The control product, placebo, consisted of a mixture of 150 mM sodium chloride and 15 mM sodium citrate. Placebo was presented in clear glass cartridges and was injected IM (deltoid [preferred site] or anterolateral quadriceps [alternate site]), followed immediately by EP with the CELLECTRA™ 5PSP device. The lot number of placebo used in the clinical trial was 1622-066.

### Duration of Treatment:

Treatment was administered over 12 weeks. The first clinical trial treatment was administered on Day 0, the second at Week 4, and the third (final) at Week 12.

### Estimands and Intercurrent Events:

Estimands and intercurrent events were not defined in the clinical trial protocol or in the statistical analysis plan (SAP).

### Statistical Methods:

### Sample Size

A total of 198 subjects were planned to be randomized to receive either 6 mg VGX-3100 or placebo IM followed by EP in a 2:1 ratio. This sample size provided 90% power to declare VGX-3100 superior to placebo, assuming the true proportion of subjects who achieved the primary endpoint was 35% and 14% for VGX-3100 and placebo, respectively. These proportions also incorporated missing data (˜10%) classified as nonregressors (failures). The assumptions were based on the Phase 2 clinical trial results.

### Analysis Sets

The analysis sets were defined as follows:

Intent-to-Treat (ITT) Set: The ITT set included all subjects who were randomized. Subjects in this sample were grouped to treatment as randomized. The ITT set was used for the primary analysis of efficacy in this clinical trial. Missing data was considered as nonregressors (failures) for the ITT efficacy analysis. A subject's regression outcome was missing if her CIN grade and HPV clearance for the Week 36 timeframe could not be determined. The ITT set was also used for summaries of demographics, baseline characteristics, disposition, and protocol deviations.

Modified Intent-to-Treat (mITT) Set: The mITT set included all subjects who received at least one (1) dose of clinical trial treatment and who had the analysis endpoint of interest. Subjects in this sample were grouped to treatment as randomized. Analysis of the mITT set was considered supportive for the corresponding ITT set for the analysis of efficacy and also served as sensitivity analyses regarding missing data.

Per-Protocol (PP) Set: The PP set was comprised of subjects who received all doses of clinical trial treatments, had no protocol violations, and had the analysis endpoint of interest. Subjects in this sample were grouped to treatment as randomized. Analyses on the PP set was considered supportive of the corresponding ITT set for the analysis of efficacy. Additional efficacy analyses on the PP set utilized the Week 36 timeframe result regardless of any procedure performed before the Week 36 timeframe, thus serving as sensitivity analyses regarding early intervention. Subjects excluded from the PP set were identified and documented prior to unblinding of the clinical trial database.

Safety Set: The safety set included all subjects who received at least one (1) dose of clinical trial treatment. Subjects were analyzed as to the treatment they actually received.

### Efficacy Analyses

### Main Analysis of Primary Efficacy Endpoint

The primary efficacy endpoint was no evidence of cervical HSIL (i.e., no evidence of CIN2 and CIN3) on histology (i.e., biopsies or excisional treatment) and no evidence of HPV-16 and/or HPV-18 in cervical samples by type-specific HPV testing at the Week 36 timeframe.

The primary hypothesis of superiority was: H0:δ≤0 vs. H1:δ>0, where δ=Pᵥ−Pₚ, and Pᵥ and Pₚ denoted the true population probabilities of the primary endpoint for VGX-3100 and placebo, respectively. The proportion in each treatment group was calculated by the number of responders divided by the total number of responders and nonresponders in the clinical trial population of the corresponding treatment group.

For the primary endpoint, Week 36 histology was evaluated based on the first biopsy or surgical excision procedure on or after Day 238; in case of multiple results on the same day, the one with worst grade was to be used. The virology result used for analysis was to be the latest result that was on or before the same date as the histology result and was taken on or after Day 238. If a subject underwent excision or cervical biopsy at any time on or after Day 1 and before Day 238, the subject was considered as nonresponder.

Number of responders and nonresponders, proportion of responders in each treatment group, and difference of proportions between the two (2) treatment groups were presented. A p-value of superiority based on a test of risk difference and corresponding 95% confidence interval (CI) using the method of Miettinen and Nurminen were computed. Superiority was concluded if the one-sided p-value was <0.025 and the corresponding lower bound of the 95% CI exceeded zero (0).

Responder proportion was also summarized based on the stratification factors separately.

Analyses of primary efficacy endpoint with the mITT and PP set served as sensitivity analyses and were considered supportive of the corresponding analysis with the ITT set.

### Secondary Efficacy Endpoint Analyses

The secondary efficacy endpoint analyses methods were the same as those for primary efficacy endpoint analysis, including the sensitivity analyses, but no p-values were computed for the secondary efficacy endpoint analyses.

### Immunogenicity Analyses

The immunogenicity analyses were performed on the mITT set with at least one (1) immunogenicity measurement.

Levels of serum anti-HPV-16 and anti-HPV-18 antibody concentrations and interferon (IFN)-γ enzyme-linked immune absorbent spot (ELISpot) response magnitudes were assessed from sera and peripheral blood mononuclear cells (PBMCs) isolated from whole blood, respectively, collected at baseline, Week 15, and Week 36. Flow cytometry response magnitudes were assessed from PBMCs isolated from whole blood collected at baseline and Week 15.

All of the endpoints were summarized as continuous variables at each visit. Increases from baseline for each postbaseline visit from ELISpot and flow cytometry, and titers for each postbaseline visit from enzyme-linked immunosorbent assay (ELISA) were compared between treatment groups. These comparisons were analyzed with differences in medians and associated exact nonparametric 95% CIs.

### Exploratory Endpoints Analyses

The relationship between the histologic regression of cervical HSIL with virologic clearance at Week 36 visit (yes, no) and a) miRNA results, b) DNA methylation results, c) colposcopy results, d) cytology results, e) HPV results, and f) baseline immunoscore results (tissue-based score derived using immunologic markers) were examined using separate logistic regression models for each result, with histologic regression of cervical HSIL with virologic clearance as the response variable and each of the results and treatment group as regressor variables. Odds ratios (ORs) and corresponding 95% CIs were provided for the regressor variables. Durability, as measured by clearance of HPV-16 and/or HPV-18 infection at Weeks 62 and 88, were summarized by number and percentage of subjects with no evidence of HPV-16 and/or HPV-18 by treatment group at each visit. Specifically, the virology results at Week 62 were those between Day 420 and Day 448 and the virology results at Week 88 were those between Day 602 and Day 630. Assessment of markers including but not limited to CD8+ and FoxP3+ infiltrating cells and visualization of granulysin, perforin, CD137, CD103 and PD-L1 in cervical tissue were to be performed. These tissue response magnitudes were compared between treatment groups using a difference in means and associated t-distribution based 95% CIs for changes from baseline at each postbaseline timepoint. Subjects enrolled in US, Canada, Mexico, Germany, and UK were to complete patient-reported outcome (PRO) questionnaires (36-Item Short Form Survey [SF-36], EuroQol 5-Dimensions 5-Level [EQ-5D-5L], and two [2] additional global PRO questions assessing quality of life [QoL] after excision or biopsy). For the SF-36, section scores at each of the eight (8) domains (physical functioning, role limitations due to physical problems, bodily pain, general health, vitality, social functioning, role limitations due to emotional problems, and mental health) and score changes from baseline were summarized at each visit by treatment group. Scoring of the eight (8) SF-36 subscales was done by Quality Metric Health Outcomes™ Scoring Software 5.0. For the EQ-5D-5L, each of the five (5) domains (mobility, self-care, usual activity, pain/discomfort, anxiety/depression) and global health status score were summarized at each visit and relative to baseline by treatment group. For the two (2) additional global PRO questions assessing QoL after excision or biopsy, the time outcome (median number of days that a worsened QoL was experienced) and the binary outcomes to yes/no questions were summarized by treatment group. SF-36 scores were compared between treatment groups using exact nonparametric 95% CIs for the differences in median changes from baseline. The EQ-5D-5L scores were analyzed in the same fashion. Days of worsened QoL for the Week 40 QoL questionnaire were analyzed using an exact nonparametric 95% CI for the difference in the median number of days. The yes/no worsened QoL responses for the Week 40 QoL questionnaire were analyzed using a 95% Miettinen and Nurminen CI for the difference in proportions between treatment groups. In addition, PRO endpoints at each visit that occurred after Week 36 were summarized according to those with excision (excluding biopsy) versus those without. The subjects in the mITT set with at least one (1) postbaseline corresponding measurement were used for PRO analyses.

### Safety Analyses

All safety analyses were conducted on the safety set.

### Adverse Events (AEs)

Adverse event verbatim reported terms were coded by system organ class (SOC) and preferred term (PT) using the latest version of Medical Dictionary for Regulatory Activities (MedDRA).

Adverse event summary tables included numbers and percentages of subjects experiencing at least one (1) event by treatment group. The following AE summary tables were generated:
Overview of AEs overall, and with onset within 28 days/7 days after clinical trial treatment
Treatment-emergent AEs (TEAEs) overall, and with onset within 28 days/7 days after clinical trial treatment by SOC and PT
Treatment-emergent AEs overall, and with onset within 28 days/7 days after clinical trial treatment by dose number, SOC, and PT
Treatment-emergent AEs overall, and with onset within 28 days/7 days after clinical trial treatment by dose number, Common Toxicity Criteria for Adverse Events (CTCAE) grade, SOC, and PT
Serious TEAEs overall, and with onset within 28 days/7 days after clinical trial treatment by dose number, SOC, and PT
Treatment-emergent AEs overall, and with onset within 28 days/7 days after clinical trial treatment by dose number, relationship to investigational product (IP) and EP, SOC, and PT
Serious TEAEs overall, and with onset within 28 days/7 days after clinical trial treatment by dose number, relationship to IP and EP, SOC, and PT
Grade ≥3 TEAEs overall, and with onset within 28 days/7 days after clinical trial treatment by dose number, SOC, and PT
Grade ≥3 TEAEs overall, and with onset within 28 days/7 days after clinical trial treatment by dose number, relationship to IP and EP, SOC, and PT
Treatment-emergent AEs with action of clinical trial treatment held overall, and with onset within 28 days/7 days after clinical trial treatment by dose number, SOC, and PT
Treatment-emergent AEs with action of clinical trial treatment permanently discontinued overall, and with onset within 28 days/7 days after clinical trial treatment by dose number, SOC, and PT
Adverse events of special interest overall, and with onset within 28 days/7 days after clinical trial treatment by dose number, SOC, and PT

A subject with two (2) or more different AEs within the same level of the MedDRA term and regimen was counted only once in that level using the most extreme incident (most severe for the intensity analyses and related for the relationship to clinical trial treatment analyses).

Data listings were provided for AE, SAE, AEs leading to treatment discontinuation, AEs with CTCAE grade ≥3, AEs with onset within 28 days/7 days after clinical trial treatment, and AEs resulting in death separately.

### Incidence of Adverse Events

For the AEs with onset date within 28 days after any dose, the frequency of SOC and PT events were compared between treatment groups with risk differences and 95% CIs using the method of Miettinen and Nurminen. As this analysis used many event categories and produced many CIs, caution was to be exercised when interpreting these CIs.

A similar analysis was provided for AEs within 7 days after any dose and for AEs during the clinical trial after any dose.

### Clinical Laboratory Evaluations

Safety laboratory tests (including hematology, serum chemistry, and urinalysis) were performed at screening (within 30 days prior to Day 0) and a summary of screening information was provided by treatment group and by total.

### Vital Signs, Physical Examination, and Other Observations Related to Safety

Vital sign data was summarized at each visit by treatment group. Changes from baseline to each scheduled postbaseline visit were presented.

During physical examination, body systems were assessed as normal, abnormal, or not examined at each scheduled visit, and the percentage of subjects with abnormal physical examination findings at each timepoint were summarized by body system and by treatment group.

Electrocardiogram results were summarized by treatment group and by total, for interpretation results including normal, abnormal not clinically significant, abnormal clinically significant, and not done.

### Responder and Nonresponder Definitions for the Primary Endpoint

Responder and nonresponder definitions for the primary endpoint were to take into account both histopathologic regression of cervical HSIL and virologic (HPV-16 and/or HPV-18) clearance from cervical samples since HPV persistence is an important factor in the clinical progression of HSIL. The responder definition also excluded subjects who underwent excision or whose cervix was biopsied at any time between their initial dose and the Week 36 endpoint tissue collection. This exclusion was included to reduce the potential for artefactual increases in the treatment effect caused by removal of HSIL tissue and potentially HPV-16 and/or HPV-18 by unplanned interval biopsies. Subjects who underwent ECC only prior to Week 36 endpoint collection were considered for responder analysis.

To qualify as a responder, the subject must have had:
1. An acceptable histology specimen at Week 36, which was interpretable by the independent PAC, and
2. An acceptable HPV ThinPrep™ sample at Week 36, with an associated valid HPV-testing result.

A responder was defined as a subject:
1. With no histologic evidence of cervical HSIL at the Week 36 evaluation
2. With no evidence of HPV-16 and/or HPV-18 at the Week 36 evaluation
3. Who did not have an unscheduled excision or biopsy sample obtained between initial dose and the Week 36 evaluation.

Conversely, a nonresponder was defined as a subject with:
1. Histologic evidence of cervical HSIL, AIS, or cervical carcinoma at the Week 36 evaluation, OR
2. Evidence of HPV-16 or HPV-18 at the Week 36 evaluation, OR
3. An excision or biopsy sample obtained between initial dose and the Week 36 evaluation, OR
4. Lack of either an acceptable Week 36 histology specimen or HPV ThinPrep™ sample. No evidence of HSIL was defined by histology as negative, squamous atypia, or LSIL. The histopathologic efficacy timeframe was defined by those who had undergone a biopsy or surgical excision at any time starting from 14 days prior to the protocol-specified target date of Week 36. The first tissue removal sample in this timeframe determined the histology endpoint. The most recent HPV clearance result prior to tissue removal, which included results from the same date, within the timeframe, determined the HPV clearance endpoint.

### Responder and Nonresponder Definitions for the Secondary Endpoints

The responder and nonresponder definitions for the secondary endpoint of histopathologic regression of cervical HSIL are detailed in Table 4.

TABLE 5
Responder and Nonresponder Definitions for the Secondary Endpoint of
Complete Histopathologic Regression of Cervical HSIL

| Responder | Nonresponder |
|---|---|
| Subject with no histologic | Subject with histologic evidence |
| evidence of cervical HSIL | of cervical HSIL, |
| squamous atypia, or LSIL | squamous atypia, LSIL, AIS, |
| at Week 36 visit^{a} | or cervical carcinoma |
| AND | at Week 36 visit |
| Subject in whom an excision | OR |
| or biopsy sample^{b} was | Subject in whom an excision or |
| NOT obtained between initial | biopsy sample^{b} was |
| dose up to Week 36 | obtained between initial |
| visit | dose up to Week 36 visit |
| | OR |
| | Subjects with no Week 36 |
| | visit sample result |

Abbreviations: AIS: Adenocarcinoma in situ; ECC: Endocervical Curettage; HSIL: High-Grade Squamous Intraepithelial Lesion; LSIL: Low-Grade Squamous Intraepithelial Lesion.
^{a}The efficacy timeframe was defined for those subjects who had undergone a biopsy or surgical excision at any time starting from 14 days prior to the protocol-specified target date of Week 36. The first tissue removal sample within the timeframe determined the histology endpoint.
^{b}Excluded ECC-only samples.

The responder and nonresponder definitions for the secondary endpoint of histopathologic nonprogression of cervical HSIL are detailed in Table 6.

TABLE 6
Responder and Nonresponder Definitions for the Secondary Endpoint of
Histopathologic Nonprogression of Cervical HSIL

| Responder | Nonresponder |
|---|---|
| Subject with no histologic | Subject with histologic |
| evidence of a worsening | evidence of worsening of |
| of cervical condition at Week 36 | cervical condition at Week 36 |
| visit^{a} relative to baseline | visit relative to baseline |
| AND | OR |
| Subject in whom an excision | Subject in whom an excision |
| or biopsy sample^{b} was | or biopsy sample^{b} was |
| NOT obtained between | obtained between initial |
| initial dose up to Week 36 | dose up to Week 36 visit |
| visit | OR |
| | Subjects with no Week 36 |
| | visit sample result |

Abbreviations: ECC: Endocervical Curettage; HSIL: High-Grade Squamous Intraepithelial Lesion.
^{a}The efficacy timeframe was defined for those subjects who had undergone a biopsy or surgical excision at any time starting from 14 days prior to the protocol-specified target date of Week 36. The first tissue removal sample within the timeframe determined the histology endpoint.
^{b}Excluded ECC-only samples.

The responder and nonresponder definitions for the secondary endpoint of complete histopathologic regression of cervical HSIL and virologic clearance of HPV are detailed in Table 7.

TABLE 7
Responder and Nonresponder Definitions for the Secondary Endpoint of
Complete Histopathologic Regression of Cervical HSIL and Virologic
Clearance of HPV

| Responder | Nonresponder |
|---|---|
| Subject with no histologic | Subject with histologic |
| evidence of cervical | evidence of cervical HSIL, |
| HSIL, squamous atypia, | squamous atypia, LSIL AIS, |
| or LSILª at Week 36 visit | cervical carcinoma at |
| and no evidence of HPV-16 | Week 36 visit |
| and/or HPV-18 at | OR |
| Week 36 visit^{b} | Subject with evidence of HPV-16 |
| AND | or HPV-18 at Week 36 visit |
| Subject in whom an excision | OR |
| or biopsy sample^{c} was | Subject in whom an excision |
| NOT obtained between initial | or biopsy sample^{c} was |
| dose up to Week 36 | obtained between initial |
| visit | dose up to Week 36 visit |
| | OR |
| | Subjects with no Week 36 |
| | visit sample result |

Abbreviations: AIS: Adenocarcinoma in situ; ECC: Endocervical Curettage; HPV: Human Papilloma Virus; HSIL: High-Grade Squamous Intraepithelial Lesion; LSIL: Low-Grade Squamous Intraepithelial Lesion.
^{a}No evidence of HSIL was defined by histology as negative, squamous atypia, or LSIL
^{b}The efficacy timeframe was defined for those subjects who had undergone a biopsy or surgical excision at any time starting from 14 days prior to the protocol-specified target date of Week 36. The first tissue removal sample within the timeframe determined the histology endpoint. The most recent HPV clearance result prior to tissue removal, which included results from the same date within the timeframe determined the HPV clearance endpoint.
^{c}Excluded ECC-only samples.

### Summary of Results

Subject Disposition: Subject disposition is summarized in Table 8.

TABLE 8
Subject Disposition (ITT Population)

| | VGX-3100 + EP | Placebo + EP | Total |
|---|---|---|---|
| | (N = 138) | (N = 63) | (N = 201) |
| | n (%) | n (%) | n (%) |
| Total number of subjects | 138 | 63 | 201 |
| randomized | | | |
| Completed all | 129 (93.5) | 61 (96.8) | 190 (94.5) |
| electroporation/trial | | | |
| treatment | | | |
| Discontinued | 7 (5.1) | 2 (3.2) | 9 (4.5) |
| electroporation/trial | | | |
| treatment | | | |
| Completed trial | 117 (84.8) | 56 (88.9) | 173 (86.1) |
| Discontinued trial | 21 (15.2) | 7 (11.1) | 28 (13.9) |
| Primary reason for | | | |
| discontinuation from trial | | | |
| treatment | | | |
| Withdrawal by subject | 1 (0.7) | 1 (1.6) | 2 (1.0) |
| Unrelated to trial | 0 | 0 | 0 |
| procedures | | | |
| Subject refused further EP | 1 (0.7) | 1 (1.6) | 2 (1.0) |
| Adverse event | 0 | 0 | 0 |
| Progressive disease | 2 (1.4) | 0 | 2 (1.0) |
| Lost to follow-up | 1 (0.7) | 0 | 1 (0.5) |
| Physician decision | 0 | 0 | 0 |
| Trial terminated | 0 | 0 | 0 |
| by sponsor | | | |
| Protocol deviation | 1 (0.7) | 0 | 1 (0.5) |
| Pregnancy | 2 (1.4) | 0 | 2 (1.0) |
| Other | 0 | 1 (1.6) | 1 (0.5) |
| Primary reason for | | | |
| discontinuation from trial | | | |
| Withdrawal by subject | 5 (3.6) | 3 (4.8) | 8 (4.0) |
| Unrelated to trial | 4 (2.9) | 3 (4.8) | 7 (3.5) |
| procedures | | | |
| Subject refused further EP | 1 (0.7) | 0 | 1 (0.5) |
| Adverse event | 1 (0.7) | 0 | 1 (0.5) |
| Progressive disease | 1 (0.7) | 0 | 1 (0.5) |
| Lost to follow-up | 8 (5.8) | 2 (3.2) | 10 (5.0) |
| Physician decision | 1 (0.7) | 0 | 1 (0.5) |
| Trial terminated | 0 | 0 | 0 |
| by sponsor | | | |
| Protocol deviation | 2 (1.4) | 0 | 2 (1.0) |
| Pregnancy | 0 | 0 | 0 |
| Other | 3 (2.2) | 2 (3.2) | 5 (2.5) |

Note:
There were two (2) subjects who were randomized to VGX-3100 + EP group but not treated.
Note:
Denominator used in the percentage calculations was randomized subjects in each treatment.
Abbreviations: EP: Electroporation; ITT: Intent-to-Treat; N: Sample Size for the Group; n: Number of Subjects.

### Demography and Baseline Characteristics

The mean age of the subjects was 31.5 years and ranged from 20 to 55 years. Majority of the subjects (77.1%) were White, and not Hispanic or Latino (82.6%). The mean BMI was 25.07 kg/m2 and ranged from 16.5 to 56.5 kg/m2. The demographic characteristics were similar across both the treatment groups.

Randomization was stratified at baseline based on age, BMI, and the CIN stage. In each individual strata and combinations of strata, the proportions of subjects were similar between the treatment groups.

Sixteen subjects (8.0%) were exposed to prophylactic HPV vaccine before clinical trial participation: 14 subjects (10.1%) in the VGX-3100+EP group and 2 subjects (3.2%) in the placebo+EP group. The most recent Pap smear result was abnormal in majority (76.6%) of subjects. The most recent Pap smears were obtained between 21 and 735 days prior to baseline. Most subjects had either CIN2 (43.9%) or CIN3 (39.8%) diagnosis. The median time from initial CIN diagnosis was 86.5 days and ranged from 21 to 4695 days. Fourteen subjects (7.0%) had received prior treatment for CIN, most commonly LEEP (11 subjects [78.6%]). The median time from last CIN treatment was 489.0 days and ranged from 250 to 4771 days.

The ITT set included all subjects who were randomized. Subjects in this sample were grouped to treatment as randomized. The ITT set was used for the primary analysis of efficacy in this clinical trial. Missing data was considered as nonregressors (failures) for the ITT efficacy analysis. A subject's regression outcome was missing if her CIN grade and HPV clearance for the Week 36 timeframe could not be determined. The ITT set was also used for summaries of demographics, baseline characteristics, disposition, and protocol deviations.

The mITT set included all subjects who received at least one (1) dose of clinical trial treatment and who had the analysis endpoint of interest. Subjects in this sample were grouped to treatment as randomized. Analysis of the mITT set was considered supportive for the corresponding ITT set for the analysis of efficacy and also served as sensitivity analyses regarding missing data.

The PP set was comprised of subjects who received all doses of clinical trial treatments, had no protocol violations, and had the analysis endpoint of interest. Subjects in this sample were grouped to treatment as randomized. Analyses on the PP set was considered supportive of the corresponding ITT set for the analysis of efficacy. Additional efficacy analyses on the PP set utilized the Week 36 timeframe result regardless of any procedure performed before the Week 36 timeframe, thus serving as sensitivity analyses regarding early intervention. Subjects excluded from the PP set were identified and documented prior to unblinding of the clinical trial database.

The safety set included all subjects who received at least one (1) dose of clinical trial treatment. Subjects were analyzed as to the treatment they actually received.

TABLE 9
Analysis Sets

| | VGX-3100 + EP | Placebo + EP | Total |
|---|---|---|---|
| Analysis Sets | (N = 138) | (N = 63) | (N = 201) |
| Intent-to-Treat (ITT) | 138 | 63 | 201 |
| Modified ITT (mITT) | 134 (97.1) | 63 (100) | 197 (98.0) |
| Per Protocol | 124 (89.9) | 60 (95.2) | 184 (91.5) |
| Safety | 136 (98.6) | 63 (100) | 199 (99.0) |

Note:
ITT set included all subjects who were randomized. mITT set included all subjects who received at least one (1) dose of clinical trial treatment based on those who had evaluation of any analysis endpoint of interest. Per Protocol set included subjects who had received all doses of clinical trial treatment and had no protocol deviations based on those who had evaluation of any analysis endpoint of interest. Safety set included subjects who received at least one (1) dose of clinical trial treatment.
Note
that subjects who received a completely opposite treatment regimen from assigned and subjects who received a mixed regimen of treatments were included in the column to which the subjects were randomized, but these subjects were analyzed for safety outcomes according to the treatment they actually received.
Abbreviations: EP: Electroporation; N: Sample Size for the Group.

### Efficacy Results

Overall, the percentage of responders was higher in the VGX-3100+EP group as compared with placebo+EP group for the primary endpoint of histopathological regression of cervical HSIL and virologic clearance of HPV-16 and/or HPV-18 at Week 36 for ITT, mITT, and PP Populations. The percentage of primary endpoint responders was significantly higher (p value <0.025) in the VGX-3100+EP group as compared with placebo+EP group in the mITT and PP Populations. Results were similar for the secondary endpoint of histopathological regression of cervical HSIL at Week 36. For other secondary endpoints including virologic clearance of HPV 16 and/or HPV-18 at Week 36; histopathological regression of cervical HSIL to normal and virologic clearance of HPV-16 and/or HPV-18 at Week 36; histopathological regression of cervical HSIL to normal at Week 36; and virologic clearance of HPV-16 and/or HPV-18 from noncervical anatomic locations at Week 36, the percentage of responders was higher in the VGX 3100 +EP group as compared with placebo+EP group, and the lower bound of the 95% CI generally exceeded zero (0). The efficacy results are summarized below.

### Primary Efficacy Endpoint

The primary endpoint was no evidence of cervical HSIL on histology (i.e., biopsy or excisional treatment) and no evidence of HPV-16 and/or HPV-18 in cervical samples by type-specific HPV testing at Week 36 visit.

In the ITT Population, the percentage of responders (i.e., subjects with histopathological regression of cervical HSIL and virologic clearance of HPV-16 and/or HPV-18 at Week 36) was 22.5% in the VGX-3100+EP group as compared with 11.1% in the placebo+EP group. The difference between the responders in the two (2) groups was not statistically significant (one-sided p value=0.029).

In the mITT Population, 31 subjects (23.7%) in the VGX-3100+EP group and 7 subjects (11.3%) in the placebo+EP group were considered responders. The difference between the responders in the two (2) groups was statistically significant (one-sided p value=0.022), indicating that VGX-3100+EP was superior to placebo+EP in achieving histopathological regression of cervical HSIL and virologic clearance of HPV-16 and/or HPV 18 at Week 36 in the mITT Population.

In the PP Population, 30 subjects (24.6%) in the VGX-3100+EP group and 7 subjects (11.7%) in the placebo+EP group were considered responders. The difference between the responders in the two (2) groups was statistically significant (one-sided p-value=0.021), indicating that VGX 3100+EP was superior to placebo+EP in achieving histopathological regression of cervical HSIL and virologic clearance of HPV-16 and/or HPV 18 at Week 36 in the PP Population. The results of the PP Population and the sensitivity analysis in PP Population supported the mITT Population results.

### Secondary Efficacy Endpoints

Histopathological regression of cervical HSIL at Week 36: In the ITT Population, the percentage of responders (i.e., subjects with histopathological regression of cervical HSIL at Week 36) was 31.9% in the VGX-3100+EP group as compared with 19.0% in the placebo+EP group. For the mITT Population, the percentage of responders was 33.6% in the VGX-3100+EP group as compared with 19.4% in the placebo+EP group and the lower bound of the 95% CI for the difference in percentage of responders exceeded zero (0), indicating superior efficacy of VGX-3100+EP in achieving histopathological regression of cervical HSIL at Week 36 in mITT Population. The results of the PP Population and the sensitivity analysis on PP Population supported the mITT Population results.

Virologic clearance of HPV-16 and/or HPV-18 at Week 36: For the ITT Population, the percentage of responders was higher in the VGX-3100+EP group (34.1%) as compared with the placebo+EP group (15.9%). The lower bound of the 95% CI for the difference in percentage of responders exceeded zero (0), indicating superior efficacy of VGX-3100+EP in achieving virologic clearance of HPV-16 and/or HPV-18 at Week 36. The results of the mITT and PP Populations and the sensitivity analysis on PP Population supported the ITT Population results.

Histopathological regression of cervical HSIL to normal and virologic clearance of HPV-16 and/or HPV-18 at Week 36: For the ITT Population, the percentage of responders was higher in the VGX-3100+EP group (18.1%) as compared with the placebo+EP group (6.3%). The lower bound of the 95% CI for the difference in percentage of responders exceeded zero (0), indicating superior efficacy of VGX-3100+EP in achieving histopathological regression of cervical HSIL to normal and virologic clearance of HPV-16 and/or HPV-18 at Week 36. The results of the mITT and PP Populations and the sensitivity analysis on PP Population supported the ITT Population results.

Histopathological regression of cervical HSIL to normal at Week 36: For the ITT Population, the percentage of responders was higher in the VGX-3100+EP group (24.6%) as compared with the placebo+EP group (11.1%). The lower bound of the 95% CI for the difference in percentage of responders exceeded zero (0), indicating superior efficacy of VGX-3100+EP in achieving histopathological regression of cervical HSIL to normal at Week 36. The results of the mITT and PP Populations and the sensitivity analysis on PP Population supported the ITT Population results.

Nonprogression of cervical HSIL to cervical carcinoma at Week 36: For the ITT Population, the percentage of responders in the VGX-3100+EP group (84.1%) was similar to those in the placebo+EP group (85.7%). The 95% CI for the difference in percentage of responders did not exclude zero (0), indicating no difference in the efficacy of VGX-3100+EP and placebo+EP in preventing progression of cervical HSIL to cervical carcinoma at Week 36. The results of the mITT and PP Populations and the sensitivity analysis on PP Population supported the ITT Population results.

Virologic clearance of HPV-16 and/or HPV-18 from noncervical anatomic locations at Week 36: In the ITT Population, the percentage of responders (i.e., subjects with virologic clearance of HPV-16 and/or HPV-18 from noncervical anatomic locations at Week 36) was 20.3% in the VGX-3100+EP group as compared with 9.5% in the placebo+EP group. In the mITT Population, the percentage of responders was 24.8% in the VGX-3100+EP group as compared with 10.7% in the placebo+EP group. The lower bound of the 95% CI for the difference in percentage of responders in the mITT Population exceeded zero (0), indicating superior efficacy of VGX-3100+EP in achieving virologic clearance of HPV-16 and/or HPV-18 from noncervical anatomic locations at Week 36 in mITT Population. The results of the PP Population supported the mITT Population results.

### Subgroup Analysis

Subgroup analyses of the primary and secondary efficacy endpoints were conducted by history of exposure to prophylactic HPV vaccines (yes, no). Histopathological regression of cervical HSIL and virologic clearance of HPV-16 and/or HPV-18 at Week 36 were also assessed by stratification factors.

Among subjects with no history of exposure to prophylactic HPV vaccine, VGX-3100+EP showed superior efficacy as compared with placebo+EP in the following efficacy measures:
Histopathological regression of cervical HSIL and virologic clearance of HPV-16 and/or HPV-18 at Week 36
Virologic clearance of HPV-16 and/or HPV-18 at Week 36
Histopathological regression of cervical HSIL to normal and virologic clearance of HPV-16 and/or HPV-18 at Week 36
Histopathological regression of cervical HSIL to normal at Week 36
Virologic clearance of HPV-16 and/or HPV-18 from noncervical anatomic locations at Week 36.

The percentage of responders for VGX-3100+EP and placebo+EP was similar with respect to following efficacy measures, irrespective of previous exposure to prophylactic HPV vaccine:
Histopathological regression of cervical HSIL at Week 36
Nonprogression of cervical HSIL to cervical carcinoma at Week 36.

The percentage of subjects with histopathological regression of cervical HSIL and virologic clearance of HPV-16 and/or HPV-18 at Week 36 was higher in the VGX-3100+EP group as compared with the placebo+EP group for all stratification combinations. The difference in percentage of responders was highest (5.8%) in subjects who were ≥25 years of age and had BMI ≤25 kg/m² and CIN2.

### Exploratory Efficacy Endpoints

### Overall Summary

The impact of prior miRNA and DNA methylation, colposcopy, cytology, and HPV result, baseline immunoscore, and baseline biomarker status on histopathologic regression of cervical HSIL and virologic clearance of HPV-16 and/or HPV-18 at Week 36 was evaluated. At the individual subject level, Day 0 and Week 8 miRNA and Day 0 and Week 15 DNA methylation values did not significantly influence the odds of the response, except the Day 0 methylated NKAIN2, for which the odds of response decreased as the Day 0 NKAIN2 increased. The odds of achieving a response at Week 36 were 3.55 times higher if the Week 15 colposcopy result showed an improvement as compared with no change (95% CI:1.69, 7.48) and 2.93 times higher if the Week 28 colposcopy result showed an improvement as compared with no change (95% CI:1.40, 6.13). The odds of achieving a response at Week 36 were 2.24 times higher if the Week 15 cytology result showed an improvement as compared with no change (95% CI:1.01, 4.99) and 9.78 times higher if the Week 28 cytology result showed an improvement as compared with possible progression (95% CI:1.23, 77.92). The odds (95% CI) of achieving a response at Week 36 were 7.93 (2.92, 21.54), 10.65 (4.39, 25.86), and 27.83 (10.64, 72.76) times higher if HPV had cleared at Weeks 8, 15, and 28, respectively, as compared with not cleared at these timepoints, indicating that clearance of HPV was associated with a response, and the response rate improved with time. Baseline immunoscore did not influence the odds of achieving a response (odds ratio [95% CI]:0.99 [0.63, 1.56]). VGX-3100+EP demonstrated superior efficacy for causing histopathological regression of cervical HSIL and virologic clearance of HPV-16 and/or HPV-18 at Week 36 in subjects with baseline biomarker status positive when compared with placebo+EP but did not have the same impact in subjects with baseline biomarker status negative.

The percentage of subjects with no evidence of cervical HPV-16 and/or HPV-18 at Weeks 62 and 88 was higher in the VGX-3100+EP group as compared with the placebo+EP group; however, at Week 88, the difference between the groups had reduced.

Patient-reported outcome measures (SF-36, EQ-5D-5L, Week 40 QoL responses) were overall similar in the VGX-3100+EP and placebo+EP groups.

Impact of Baseline Biomarker Status on Histopathologic Regression of Cervical HSIL and Virologic Clearance of HPV-16 and/or HPV-18 at Week 36

Impact of baseline biomarker status on histopathologic regression of cervical HSIL and virologic clearance of HPV-16 and/or HPV-18 at Week 36 is summarized for the ITT Population in Table 10.

Biomarker status was determined based on a combination of 12 miRNAs: hsa.miR.576.5p, hsa.miR.143.3p, hsa.miR185.5p, hsa.let.7b.5p, hsa.miR.23a.3p, hsa.miR.25.3p, hsa.miR.190b.5p, hsa.miR.145.5p, hsa.miR181a.5p, hsa.miR.625.5p, hsa.miR744.5p, and hsa.miR.206. These 12 miRNAs were then used to train a classification model that generated parameters enabling the creation of a final model used to define individual subjects as, “biomarker-positive” or “biomarker-negative”. More information regarding the model(s) used and the selection of miRNAs for final definition of “positive vs. negative” are outside of the scope of this CSR.

Among subjects with baseline biomarker status positive, 66.0% subjects in the VGX-3100+EP group and 15.0% subjects in the placebo+EP group were considered responders. The difference in percentage of responders (95% CI) was 51.0 (25.9, 67.9). The lower bound of the 95% CI exceeded zero (0), indicating superior efficacy of VGX-3100+EP for causing histopathological regression of cervical HSIL and virologic clearance of HPV-16 and/or HPV-18 at Week 36 in subjects with baseline biomarker status positive.

Among subjects with baseline biomarker status negative, zero (0) subjects in the VGX-3100+EP group and 9.8% subjects in the placebo+EP group were considered responders. The difference in percentage of responders (95% CI) was −9.8 (−22.6, −3.8). The upper bound of the 95% CI was less than zero (0), indicating that VGX-3100+EP did not have the same impact in subjects with baseline biomarker status negative as in subjects with baseline biomarker status positive.

TABLE 10
Histopathological Regression of Cervical HSIL and Virologic Clearance
of HPV-16 and/or HPV-18 at Week 36 by Baseline Biomarker Status
(ITT Population)

| | | | Difference in |
|---|---|---|---|
| | VGX-3100 | Placebo | Percentage |
| | + | + | (95% CI) |
| | EP | EP | (VGX-3100 + EP) − |
| | (N = 138) | (N = 63) | (Placebo + EP) |
| Overall responders, n (%)^{a} | 31 (22.5) | 7 (11.1) | 11.4 (−0.4, 21.2) |
| Baseline biomarker status^{b} | | | |
| Biomarker-positive | 47 | 20 | |
| Responders, n (%)^{a} | 31 (66.0) | 3 (15.0) | 51.0 (25.9, 67.9) |
| Biomarker-negative | 90 | 41 | |
| Responders, n (%)^{a} | 0 | 4 (9.8) | −9.8 (−22.6, −3.8) |
| Biomarker not determined | 1 | 2 | |
| Responders, n (%)^{a} | 0 | 0 | 0 (NE, NE) |

Note:
Denominator used in the percentage calculations in the section of overall was the number of subjects who were randomized. Denominator used in the percentage calculations in the section of baseline biomarker status was number of subjects corresponding to biomarker-positive, biomarker-negative, and biomarker not determined, respectively.
Abbreviations: CI: Confidence Interval; EP: Electroporation; HPV: Human Papilloma Virus; HSIL: High-Grade Squamous Intraepithelial Lesion; ITT: Intent-to-Treat; N: Sample Size for the Group; n: Number of Subjects; NE: Not Estimated.
^{a}A responder was defined as a subject with: 1) no histologic evidence of cervical HSIL and 2) no evidence of HPV-16 and/or HPV-18 at the Week 36 timeframe. Also, a subject who underwent excision or whose cervix was biopsied at any time on or after her initial dose and before her Week 36 timeframe was considered a nonresponder. The histopathologic efficacy timeframe was defined by biopsy or surgical excision at any time starting from 14 days prior to the protocol-specified target date of Week 36. The first tissue removal sample within the timeframe determined the histology endpoint. The most recent HPV clearance result prior to tissue removal, which included results from the same date within the timeframe, determined the HPV clearance endpoint.
^{b}Biomarker status was determined based on a combination of 12 microRNAs: hsa.miR.576.5p, hsa.miR.143.3p, hsa.miR185.5p, hsa.let.7b.5p, hsa.miR.23a.3p, hsa.miR.25.3p, hsa.miR.190b.5p, hsa.miR.145.5p, hsa.miR181a.5p, hsa.miR.625.5p, hsa.miR744.5p and hsa.miR.206.

### Immunogenicity Results:

The following are the immunogenicity results.
VGX-3100 was immunogenic as seen from the geometric means of the reciprocal endpoint titers, which were several-fold higher in the VGX-3100+EP group as compared with the placebo+EP group at Weeks 8, 15, and 36 for both HPV-16 E7 and HPV-18 E7. Anti-HPV-16 E6 and anti-HPV-18 E6 antibodies were not assayed.
An increase from baseline was seen in the spot forming units (SFU) per 10⁶ PBMCs of HPV-16 E6, HPV-16 E7, HPV-18 E6, HPV-18 E7, and related combinations in the VGX-3100+EP group as compared with the placebo+EP group at all postbaseline timepoints (Weeks 8, 15, and 36).
The median increases in CEF and PMA levels appeared to be similar in both treatment groups at Weeks 8, 15, and 36.
All parameters including CD8+CD137+perforin+, CD8+CD38+perforin+, and CD8+CD69+perforin+showed an increase in the VGX-3100+EP group indicating greater cellular immune responses (as measured by activated CD8+T cells with lytic potential) on flow cytometry as compared with the placebo+EP group at Week 15.
Changes from baseline to Week 36 in CD8+, CD103+, FoxP3+, and perforin+cells in cervical tissue normal epithelium, normal stroma, CIN2/3 epithelium, and CIN2/3 stroma were small and generally similar between the treatment groups.

### Safety Results:

Overall, IM injection of VGX-3100 or placebo followed by EP was well-tolerated by subjects with HPV-16 and/or HPV-18 associated HSIL of cervix. The safety results are summarized below.
Through the clinical trial completion, the incidence of TEAEs was similar among the 2 treatment groups.
A total of 96.3% subjects in the VGX-3100+EP group and 98.4% subjects in the placebo+EP group reported at least one (1) TEAE.
Treatment-emergent AEs (TEAEs) in 9.6% subjects in the VGX-3100+EP group and 9.7% subjects in the placebo+EP group were considered to be serious. Most treatment-emergent SAEs were reported in the SOC of Neoplasms Benign, Malignant and Unspecified (including Cysts and Polyps). None of the SAEs were related to IP or EP. One (1) subject died during the clinical trial on Day 450, 365 days after Dose 3 of VGX-3100+EP due to the TEAE of pulmonary embolism.
Overall, injection-site pain, headache, fatigue, injection-site erythema, injection-site pruritus, myalgia, and injection-site swelling were the most commonly reported TEAEs during the clinical trial, and the incidences of these TEAEs in VGX-3100+EP and placebo+EP groups, respectively, were as follows: Injection-site pain was reported in 78.7% and 80.6% subjects; headache was reported in 33.1% and 30.6% subjects; fatigue was reported in 28.7% and 27.4% subjects; injection-site erythema and injection-site pruritus each were reported in 25.0% and 22.6% subjects; myalgia was reported in 21.3% and 24.2% subjects; and injection-site swelling was reported in 20.6% and 24.2% subjects.
The incidence of most TEAEs did not differ per number of doses received by the subject. Few TEAEs were reported more frequently after Dose 1 in both VGX-3100+EP and placebo+EP groups and their incidence generally decreased after Dose 2 and Dose 3. These TEAEs included nausea, fatigue, injection-site pain, injection-site swelling, malaise, myalgia, and headache. The incidences of injection-site erythema and injection-site pruritis decreased after Dose 1 in the placebo+EP group, but slightly increased after Dose 1 in the VGX-3100+EP group. Most of these TEAEs were reported within 28 days of clinical trial treatment.
Most TEAEs were grade 1 or 2 in intensity, irrespective of time of onset from administration of clinical trial treatment. Treatment-emergent AEs of CTCAE grade ≥3 were reported in 14.0% subjects in the VGX-3100+EP group and 11.3% subjects in placebo+EP group. Most common TEAEs of CTCAE grade ≥3 included injection-site pain and headache.
Treatment-emergent AEs in 83.8% subjects in the VGX-3100+EP group and 90.3% subjects in the placebo+EP group were related to the IP or EP. Overall, injection-site pain, fatigue, and headache were the most commonly reported IP- or EP-related TEAEs through the clinical trial completion. Treatment-emergent AEs with CTCAE grade ≥3 severity reported by the investigator to be related to the IP, EP, or both through the clinical trial completion in >1% of subjects included injection-site pain, myalgia, and headache. All of these TEAEs were reported within 7 days of clinical trial treatment.
The TEAEs that led to withholding of clinical trial treatment included injection-site pain and upper respiratory tract infection. The TEAE of injection-site pain reported in one (1) other subject in the VGX-3100+EP group within 7 days after Dose 1 of clinical trial treatment led to permanent discontinuation of clinical trial treatment.
Adverse pregnancy outcomes included abortion spontaneous (1 subject) and ectopic pregnancy (1 subject), both in the placebo+EP group.
   Through the clinical trial, no TEAEs related to abnormal laboratory results were reported. Vital signs and physical examination were found to be normal in most subjects and few AEs were reported due to abnormal vital signs or physical examination findings.

### Conclusions

VGX-3100+EP showed superior efficacy as compared with placebo+EP in achieving histopathological regression of cervical HSIL and virologic clearance of HPV-16 and/or HPV 18 at Week 36 and was safe and well-tolerated by subjects with HPV-16 and/or HPV-18 associated HSIL of cervix.

### Example 2

Addressing miRNA to Establish a Predictive Value of Clinical Response after VGX-3100 Treatment of Cervical HSIL

In a Phase 3 study of VGX-3100 for the treatment of HPV16 and/or HPV18-associated HSIL (designated as Study HPV-301/REVEAL1; see Example 1), miRNAs were assessed via RNA sequencing of plasma samples isolated from subjects prior to dosing with VGX-3100 to identify a signature of miRNAs that had the ability to predict clinical response in the form of cervical HSIL lesion regression concomitant with elimination of HPV16 and HPV18 infection at the efficacy assessment timepoint (Study Week 36). To achieve this, raw miRNA counts obtained from RNA sequencing were normalized to counts per million (CPM) and miRNA displaying near zero variance in the VGX-3100 treated group across all subjects irrespective of clinical outcome were removed based on their inability to convey differential information. The resultant values of miRNA reference sequences (defined as those sequences that map directly to the miRbase v22.1<www_miRNA_org>) were used to conduct a classification and regression tree (CART) (Paradiso et al., BRCA germline mutation test for all woman with ovarian cancer? BMC Cancer (2019); Talhou, et al., Confirmation of ProMisE: A simple, genomics-based clinical classifier for endometrial cancer. Cancer (2017)) and random forest analysis using the statistical computing language R (v3.6.2) (Toth, et al., Random forest-based modelling to detect biomarkers for prostate cancer progression. Clin Epigenetics. (2019); Yan et al., Identification of candidate colon cancer biomarkers by applying a random forest approach on microarray data. Oncol Rep. (2012)). Results of this work generated a predictive signature based on the construction of a random forest named “RF_21_20”.

Creation of “(RF_21_20” as a miRNA-based predictive marker of clinical response after treatment with VGX-3100 for Cervical HSIL. For the creation of the random forest model known as RF_21_20, microRNAs were down selected based on performance following multiple cycles of random forest generation. The top 12 miRs, as ranked by variable importance, were then used to train a random forest model (R package randomForest v4.6-14) for classification.

The classifier was trained and tested on the same dataset, with the following caveats to mitigate overfitting: MWMOTE (Majority Weighted Minority Oversample TEchnique) was used to generate 70 synthetic “responder” data points to balance the training data set. The resultant oversampled training data were used to tune several hyperparameters (mtry, ntree, and maxnodes) of the Random Forest model. Final hyperparameters were chosen based on model performance averaged from 10-fold cross validation.

Following the identification of desirable hyperparameters, a final random forest model was trained using the oversampled data set. This model (RF_21_20) was tested on the performance of the original intent-to-treat (ITT), modified intent-to-treat (mITT) and per protocol (PP) data sets (synthetic responder samples used during training were removed for testing). Additionally, no data from the Placebo data set was used in the model training process but was assessed during testing.

The model was selected for further investigation based on overall accuracy, positive predictive value (PPV; defined here as the frequency of subjects exhibiting regression of HSIL and elimination of HPV16/18 infection out of those predicted to respond per the classifier), and the number of miRNAs used to generate the model with a soft cap of 15 miRNAs allowed. Model RF_21_20 consists of 12 miRNAs across 21 trees. The 12 miRNAs are as follows:
hsa.miR.576.5p
hsa.miR.143.3p
hsa.miR.185.5p
hsa.let.7b.5p
hsa.miR.23a.3p
hsa.miR.25.3p
hsa.miR.190b.5p
hsa.miR.145.5p
hsa.miR.181a.5p
hsa.miR.625.5p
hsa.miR.744.5p
hsa.miR.206

The miRNA sequences for the twelve miRNA species listed above are those that map directly to miRBase v22.1.

Three tables listed below break out model performance in both VGX-3100 treated subjects as well as Placebo subjects with respect to positive predictive value (PPV), negative predictive value (NPV), Accuracy, Sensitivity and Specificity (as defined by the Table Legend) for the intent-to-treat (ITT; all subjects randomized), modified intent-to-treat (mITT; all randomized and evaluable subjects) and per-protocol (PP; received all three doses of study drug and was not considered a protocol violator and was evaluable) populations.

As noted in Table 11, in the ITT population, the RF_21_20 model correctly predicted a positive response to treatment with VGX-3100 (regression of HSIL and concomitant elimination of HPV16/18 infection) at a range of 65.9% to 65.2% across two pre-treatment timepoints (noted in the table as Day 0 and screen), while the Placebo population prediction metrics were 11.1% and 8.3% respectively. The difference in predictive values and the low variance for both groups (0.7% and 2.8% respectively) show that the prediction has value in the context of VGX-3100 treatment and is robust when assessing for day-to-day variation.

TABLE 11
Model RF_21_20 Performance in the ITT
population of REVEAL1/HPV-301
RF_21_20-ITT Population of REVEAL1/HPV-301

| | VGX-3100, | VGX-3100, | PLACEBO, | PLACEBO, |
|---|---|---|---|---|
| | Day 0 | Screen | Day 0 | Screen |
| POSITIVE | 65.9% | 65.2% | 11.1% | 8.3% |
| PREDICTIVE | | | | |
| VALUE | | | | |
| NEGATIVE | 100.0% | 100.0% | 92.5% | 85.3% |
| PREDICTIVE | | | | |
| VALUE | | | | |
| ACCURACY | 88.4% | 87.7% | 67.2% | 53.5% |
| SENSITIVITY | 100.0% | 100.0% | 40.0% | 28.6% |
| SPECIFICITY | 85.0% | 84.0% | 69.8% | 56.9% |

Responder = study subjects in REVEAL1/HPV-301 who met the primary endpoint of HSIL lesion regression to LSIL or lower with concomitant elimination of HPV16/HPV18 infection
Biomarker Positive = study subjects in REVEAL1/HPV-301 that the RF_21_20 model predicts to be a Responder as defined above prior to VGX-3100 or Placebo administration are considered “Biomarker Positive”
PPV = # biomarker-positive subjects who are responders/# biomarker-positive subjects
NPV = # biomarker-negative subjects who are responders/# biomarker-negative subjects
Accuracy = # biomarker-positive subjects who are responders + # biomarker-negative subjects who are non-responders/# subjects
Sensitivity = # biomarker-positive subjects who are responders/# responders
Specificity = # biomarker-negative subjects who are non-responders/# non-responders

Table 12 describes RF_21_20 model performance in the context of the mITT population in which response to treatment with VGX-3100 was correctly predicted at a range of 72.5% to 69.8% across two the pre-treatment timepoints, while the Placebo population prediction metrics were 11.1% and 8.7% respectively. As with the ITT population, the difference in predictive values and the low variance for both groups (2.7% and 2.4% respectively) show that the prediction has value in the context of VGX-3100 treatment and retains robustness when assessing for day-to-day variation.

TABLE 12
Model RF_21_20 Performance in the mITT population
of REVEAL1/HPV-301
RF_21_20-mITT Population of REVEAL1/HPV-301

| | VGX-3100, | VGX-3100, | PLACEBO, | PLACEBO, |
|---|---|---|---|---|
| | Day 0 | Screen | Day 0 | Screen |
| POSITIVE | 72.5% | 69.8% | 11.1% | 8.7% |
| PREDICTIVE | | | | |
| VALUE | | | | |
| NEGATIVE | 100.0% | 100.0% | 92.3% | 85.3% |
| PREDICTIVE | | | | |
| VALUE | | | | |
| ACCURACY | 91.1% | 89.5% | 66.7% | 54.4% |
| SENSITIVITY | 100.0% | 100.0% | 40.0% | 28.6% |
| SPECIFICITY | 88.3% | 86.2% | 69.2% | 58.0% |

Responder = study subjects in REVEAL1/HPV-301 who met the primary endpoint of HSIL lesion regression to LSIL or lower with concomitant elimination of HPV16/HPV18 infection
Biomarker Positive = study subjects in REVEAL1/HPV-301 that the RF_21_20 model predicts to be a Responder as defined above prior to VGX-3100 or Placebo administration are considered “Biomarker Positive”
PPV = # biomarker-positive subjects who are responders/# biomarker-positive subjects
NPV = # biomarker-negative subjects who are responders/# biomarker-negative subjects
Accuracy = # biomarker-positive subjects who are responders + # biomarker-negative subjects who are non-responders/# subjects
Sensitivity = # biomarker-positive subjects who are responders/# responders
Specificity = # biomarker-negative subjects who are non-responders/# non-responders

Table 13 describes RF_21_20 model performance in the context of the PP population, which has high similarities with the mITT population. In this case, response to treatment with VGX-3100 was correctly predicted at a range of 71.8% to 69.0% across two the pre-treatment timepoints, while the Placebo population prediction metrics were 11.1% and 8.7% respectively. As with the ITT and mITT populations, the difference in predictive values and the low variance for both groups (2.8% and 2.4% respectively) show that the prediction has value in the context of VGX-3100 treatment and retains robustness when assessing for day-to-day variation.

TABLE 13
Model RF_21_20 Performance in the PP population of REVEAL1/HPV-301
RF_21_20-PP Population of REVEAL1/HPV-301

| | VGX-3100, | VGX-3100, | PLACEBO, | PLACEBO, |
|---|---|---|---|---|
| | Day 0 | Screen | Day 0 | Screen |
| POSITIVE | 71.8% | 69.0% | 11.1% | 8.7% |
| PREDICTIVE | | | | |
| VALUE | | | | |
| NEGATIVE | 100.0% | 100.0% | 92.3% | 85.3% |
| PREDICTIVE | | | | |
| VALUE | | | | |
| ACCURACY | 90.4% | 88.7% | 66.7% | 54.4% |
| SENSITIVITY | 100.0% | 100.0% | 40.0% | 28.6% |
| SPECIFICITY | 87.2% | 84.9% | 69.2% | 58.0% |

Responder = study subjects in REVEAL1/HPV-301 who met the primary endpoint of HSIL lesion regression to LSIL or lower with concomitant elimination of HPV16/HPV18 infection
Biomarker Positive = study subjects in REVEAL1/HPV-301 that the RF_21_20 model predicts to be a Responder as defined above prior to VGX-3100 or Placebo administration are considered “Biomarker Positive”
PPV = # biomarker-positive subjects who are responders/# biomarker-positive subjects
NPV = # biomarker-negative subjects who are responders/# biomarker-negative subjects
Accuracy = # biomarker-positive subjects who are responders + # biomarker-negative subjects who are non-responders/# subjects
Sensitivity = # biomarker-positive subjects who are responders/# responders
Specificity = # biomarker-negative subjects who are non-responders/# non-responders

Summary of “RF_21_20” as miRNA-based predictive marker of clinical response after treatment with VGX-3100 for Cervical HSIL. The RF_21_20 model described above was created using standard methodology common for signature isolation from complex datasets13-16. RF_21_20 clearly displays the ability to predict regression of cervical HSIL and clearance of HPV16/18 infection following VGX-3100 treatment as demonstrated by the fact that accuracy, PPV, NPV, sensitivity and specificity are all superior when this model is applied to the treated group compared to placebo. In particular, the difference in PPVs across all datasets in the VGX-3100 treated group from REVEAL1HPV-301 compared to the placebo group suggest that this model is accurately able to identify women who are more likely to derive benefit from treatment with VGX-3100.

The following list of embodiments is intended to complement, rather than displace or superseded, the previous descriptions.

Embodiment 1. A method of treating human papillomavirus (HPV) type 16- or HPV type 18-related high grade cervical intraepithelial lesion (HSIL), said method comprising: (a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, hsa.miR.744.5p, and has.miR.206; (b) calculating normalized levels for each of the miRNAs and isomiRs thereof in the set; (c) determining a miRNA signature based on normalized levels of the miRNAs and isomiRs thereof in the set; (d) categorizing the subject as biomarker positive or biomarker negative based on the miRNA signature; and (e) administering a therapeutically effective amount of VGX-3100 to said subject if the subject is categorized as biomarker positive.

Embodiment 2. The method of embodiment 1, wherein the presence of the miRNA is determined by RNA sequencing.

Embodiment 3. The method of embodiment 1 or embodiment 2, wherein the biological sample is a plasma sample.

Embodiment 4. The method of any one of the preceding embodiments, wherein the plasma sample is isolated from the subject prior to administration of VGX-3100.

Embodiment 5. The method of any one of the preceding embodiments, wherein HPV type 16- or HPV type 18-related HSIL is determined by a biopsy.

Embodiment 6. The method of any one of the preceding embodiments, wherein VGX-3100 is administered to the subject by intramuscular injection followed by electroporation.

Embodiment 7. The method of any one of the preceding embodiments, wherein VGX-3100 is administered to the subject at a dose of 6 mg.

Embodiment 8. The method of any one of the preceding embodiments, wherein VGX-3100 is administered to the subject three times over the course of 12 weeks.

Embodiment 9. The method of any one of the preceding embodiments, wherein VGX-3100 is formulated at a concentration of 6 mg/ml in 150 mM sodium chloride and 15 mM sodium citrate.

Embodiment 10. The method of any one of the preceding embodiments, wherein administration of VGX-3100 results in virologic clearance of HPV-16 and/or HPV-18 and histopathologic regression of cervical HSIL.

Embodiment 11. The method of any one of the preceding embodiments, wherein administration of VGX-3100 results in histopathologic regression of cervical HSIL.

Embodiment 12. The method of any one of the preceding embodiments, wherein administration of VGX-3100 results in virologic clearance of HPV-16 and/or HPV-18.

Embodiment 13. The method of any one of the preceding embodiments, wherein administration of VGX-3100 results in complete histopathologic regression of cervical HSIL to normal.

Embodiment 14. The method of any one of the preceding embodiments, wherein administration of VGX-3100 results in complete histopathologic regression of cervical HSIL to normal and virologic clearance of HPV-16 and/or HPV-18.

Embodiment 15. The method of any one of the preceding embodiments, wherein administration of VGX-3100 results in histopathologic nonprogression.

Embodiment 16. The method of any one of the preceding embodiments, wherein administration of VGX-3100 results in clearance of HPV-16 and/or HPV-18 infection from noncervical anatomic locations.

Embodiment 17. The method of any one of the preceding embodiments, wherein administration of VGX-3100 results in improved humoral and cellular immune response to VGX-3100 following a third administration of VGX-3100 and at 36 weeks following administration of VGX-3100 as assessed relative to baseline.

Embodiment 18. The method of any one of embodiments 10 to 17, wherein the result of VGX-3100 administration is evaluated at 36 weeks following administration of VGX-3100.

What is claimed:

1. A method of treating human papillomavirus (HPV) type 16- or HPV type 18-related high grade cervical intraepithelial lesion (HSIL), said method comprising:
(a) evaluating one or more biological samples from a subject who has HPV type 16- or HPV type 18-related HSIL for the presence of a set of miRNAs and isomiRs thereof consisting of hsa.miR.576.5p, hsa.miR.143.3p, hsa.miR.185.5p, hsa.let.7b.5p, hsa.miR.23a.3p, hsa.miR.25.3p, hsa.miR.190b.5p, hsa.miR.145.5p, hsa.miR.181a.5p, hsa.miR.625.5p, hsa.miR.744.5p, and has.miR.206;
(b) calculating normalized levels for each of the miRNAs and isomiRs thereof in the set;
(c) determining a miRNA signature based on normalized levels of the miRNAs and isomiRs thereof in the set;
(d) categorizing the subject as biomarker positive or biomarker negative based on the miRNA signature; and
(e) administering a therapeutically effective amount of VGX-3100 to said subject if the subject is categorized as biomarker positive.

2. The method of claim 1, wherein the presence of the miRNA is determined by RNA sequencing.

3. The method of claim 1, wherein the biological sample is a plasma sample.

4. The method of claim 1, wherein the plasma sample is isolated from the subject prior to administration of VGX-3100.

5. The method of claim 1, wherein HPV type 16- or HPV type 18-related HSIL is determined by a biopsy.

6. The method of claim 1, wherein VGX-3100 is administered to the subject by intramuscular injection followed by electroporation.

7. The method of claim 1, wherein VGX-3100 is administered to the subject at a dose of 6 mg.

8. The method of claim 1, wherein VGX-3100 is administered to the subject three times over the course of 12 weeks.

9. The method of claim 1, wherein VGX-3100 is formulated at a concentration of 6 mg/ml in 150 mM sodium chloride and 15 mM sodium citrate.

10. The method of claim 1, wherein administration of VGX-3100 results in virologic clearance of HPV-16 and/or HPV-18 and histopathologic regression of cervical HSIL.

11. The method of claim 1, wherein administration of VGX-3100 results in histopathologic regression of cervical HSIL. 12 The method of claim 1, wherein administration of VGX-3100 results in virologic clearance of HPV-16 and/or HPV-18.

13. The method of claim 1, wherein administration of VGX-3100 results in complete histopathologic regression of cervical HSIL to normal.

14. The method of claim 1, wherein administration of VGX-3100 results in complete histopathologic regression of cervical HSIL to normal and virologic clearance of HPV-16 and/or HPV-18.

15. The method of claim 1, wherein administration of VGX-3100 results in histopathologic nonprogression.

16. The method of claim 1, wherein administration of VGX-3100 results in clearance of HPV-16 and/or HPV-18 infection from noncervical anatomic locations.

17. The method of claim 1, wherein administration of VGX-3100 results in improved humoral and cellular immune response to VGX-3100 following a third administration of VGX-3100 and at 36 weeks following administration of VGX-3100 as assessed relative to baseline.

18. The method of claim 10, wherein the result of VGX-3100 administration is evaluated at 36 weeks following administration of VGX-3100.


SEQUENCE LISTING (4 sequences)

SEQ ID NO: 1 - DNA, 818 bp, synthetic construct
  source 1..818: mol_type = other DNA
gaattcgccaccatggactggacctggatcctgttcctggtggccgccgccacacgggtgcacagcttccaggacccccaggagagcggcagaaagctgcctcagctgtgtaccgagctgcagaccaccatccacgacatcatcctggagtgtgtgtactgtaagcagcagctgctgaggagagaggtgtacgaccgggacctgtgtatcgtgtacagggacggcaatccctacgccgtgtgtgacaagtgcctgaagttctacagcaagatcagcgagtaccggcactactgctacagcctgtacggcaccaccctggagcagcagtacaacaagcccctgtgtgacctgctgatccggtgtatcaactgccagaagcccctgcagagacacctggacaagaagcagcggttccacaacatcaggggcagatggaccggcagatgtatgagctgctgccggagcagcagaaccagaagggagacccagctgagaggccggaagagaagaagccacggcgatacccccaccctgcacgagtacatgctggacctgcagcctgagaccaccgatctgtacggctacggccagctgaatgacagcagcgaggaggaggatgagatcgacggccctgccggccaggccgagcccgacagagcccactacaacatcgtgaccttttgctgtaagtgtgacagcaccctgagactgtgcgtgcagagcacccacgtggacatcagaaccctggaggatctgctgatgggcaccctgggcatcgtgtgtcccatctgctcccagaaaccctgatgagcggccgc

SEQ ID NO: 2 - DNA, 837 bp, synthetic construct
  source 1..837: mol_type = other DNA
atggactggacctggatcctgttcctggtggccgctgccacacgggtgcacagcgccagattcgaggaccccaccaggagcggctacaagctgcccgatctgtgtaccgagctgaacaccagcctgcaggacatcgagatcacctgtgtgtactgtaagaccgtgctggagctgaccgaggtgttcgagaaggacctgttcgtggtgtacagggacagcatcccccacgccgcctgccacaagtgtatcgacttctacagccggatccgggagctgagacactacagcgacagcgtgtacggcgataccctggagaagctgaccaacaccggcctgtacaacctgctgatccggtgcctgagatgccagaagcccctgctgagacacctgaacgagaagcggcggttccacaacatcgccggccactacagaggccagtgccacagctgctgtaacagggccaggcaggagagactgcagcggagaagagagacccaggtgaggggcaggaagagaagaagccacggccccaaggccaccctgcaggatatcgtgctgcacctggagccccagaatgagatccccgtggatctgctgggccacggccagctgtccgacagcgaggaggagaacgacgagatcgacggcgtgaatcaccagcacctgcctgccagaagagccgagcctcagaggcacaccatgctgtgtatgtgctgtaagtgtgaggcccggatcgaactggtggtggagagcagcgccgacgacctgagagccttccagcagctgttcctgaacaccctgagcttcgtgtgtccttggtgtgccagccagcagtga

SEQ ID NO: 3 - protein, 264 aa, synthetic construct
MDWTWILFLVAAATRVHSFQDPQESGRKLPQLCTELQTTIHDIILECVYCKQQLLRREVYDRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQLRGRKRRSHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 4 - protein, 278 aa, synthetic construct
MDWTWILFLVAAATRVHSARFEDPTRSGYKLPDLCTELNTSLQDIEITCVYCKTVLELTEVFEKDLFVVYRDSIPHAACHKCIDFYSRIRELRHYSDSVYGDTLEKLTNTGLYNLLIRCLRCQKPLLRHLNEKRRFHNIAGHYRGQCHSCCNRARQERLQRRRETQVRGRKRRSHGPKATLQDIVLHLEPQNEIPVDLLGHGQLSDSEEENDEIDGVNHQHLPARRAEPQRHTMLCMCCKCEARIELVVESSADDLRAFQQLFLNTLSFVCPWCASQQ